(19) United States
(12) Patent Application Publication
(10) Pub. No.: US 2022/0220125 A1
(43) Pub. Date: Jul. 14, 2022

(54) FUSED ISOINDOLIN-1-ONE DERIVATIVES USEFUL AS GRK2 INHIBITORS

(71) Applicant: Janssen Pharmaceutica NV, Beerse (BE)

(72) Inventor: Guozhang Xu, Chesterbrook, PA (US)

(21) Appl. No.: 17/603,961

(22) PCT Filed: Apr. 15, 2020
(86) PCT No.: PCT/EP2020/060608

§ 371 Date: Oct. 15, 2021

Related U.S. Application Data
(60) Provisional application No. 62/834,438, filed on Apr. 16, 2019.

(51) Int. Cl. C07D 498/04; C07D 487/04; C07D 471/14

(52) U.S. Cl. CPC C07D 498/04; C07D 471/14; C07D 487/04

(57) ABSTRACT

The present invention is directed to fused isoindolin-1-one derivatives, pharmaceutical compositions containing them and their use in the treatment of disorders and conditions modulated by GRK2.

### CROSS REFERENCE TO RELATED APPLICATIONS

This application claims priority to U.S. Provisional Patent Application No. 62/834,438, filed Apr. 16, 2019, the disclosure of which is hereby incorporated by reference in its entirety.

### FIELD OF THE INVENTION

The present invention is directed to fused isoindolin-1-one derivatives, pharmaceutical compositions containing them and their use in the treatment of disorders and conditions modulated by GRK2. More particularly, the compounds of the present invention are useful in the treatment of for example, cardiac failure, cardiac hypertrophy, hypertension, Type II diabetes Mellitus, NASH, NAFLD, N-stage chronic kidney disease, kidney failure, etc.

### BACKGROUND OF THE INVENTION

G-protein-coupled receptor kinase 2 (GRK2) is a G-protein-coupled receptor kinase that is ubiquitously expressed in many tissues and regulates various intracellular mechanisms. The up- or down-regulation of GRK2 correlates with several pathological disorders. GRK2 plays an important role in the maintenance of heart structure and function; thus, this kinase is involved in many cardiovascular diseases. GRK2 up-regulation can worsen cardiac ischemic; furthermore, increased kinase levels occur during the early stages of heart failure and in hypertensive subjects. GRK2 up-regulation can lead to changes in the insulin signaling cascade, which can translate to insulin resistance. Increased GRK2 levels also correlate with the degree of cognitive impairment that is typically observed in Alzheimer's disease. (GUCCIONE, M., et al., “G-Protein-Coupled Receptor Kinase 2 (GRK2) Inhibitors: Current Trends and Future Perspectives”, J. Med. Chem, 2016, pp 9277-9294, Vol 59 (20)).

GRK2 is a prototypic GRK. This cytosolic protein is ubiquitously expressed in many tissues, but it is particularly important for embryonic development and heart function. GRK2 plays a key role in several signal transduction pathways. This protein can trigger receptor desensitization and internalization through β-arrestin binding to activated GPCRs. GRK2 can also phosphorylate different effectors involved in signal transduction. Moreover, the expression and/or function of GRK2 is altered in several pathological conditions, including cardiovascular and inflammatory pathologies.

Heart failure (HF) is the most common disease for hospitalization in the elderly, with approximately 10% of men and 8% of women over the age of 60 affected. The prevalence of HF is growing with the rise of an aging population in developed countries. There remains an intense need for novel beneficial HF therapies, with more than 3 million people in the United States diagnosed per year, and HF related mortality and rehospitalization rates remaining high despite the modest improvement in survival rates seen from advances in device therapy and pharmacological therapy (angiotensin II receptor blockers, angiotensin converting enzyme inhibitors, and β-blockers). A plethora of research into HF has revealed it to be a complex disease associated with various pathogenetic mechanisms, including ventricular remodeling, excessive neurohormonal stimulation, abnormal Ca²⁺ handling, and proliferation of the extracellular matrix. Although an overstimulation of the sympathetic nervous system (SNS) initially compensates for cardiac dysfunction, the subsequent release of catecholamine ultimately promotes disease progression via long-term exposure. Activation of the SNS is mediated by adrenergic receptors (AR), and chronic β-AR activation induces β-AR desensitization and downregulation, subsequently leading to the reduction of β-AR signaling. G-protein receptor kinase (GRK) 2 phosphorylates agonist-occupied β-AR, promotes the binding of β-AR arrestin to the Gβγ subunit of the G-protein, facilitates the G-protein uncoupling from β-AR, and results in β-AR desensitization and downregulation. In the hearts of HF patients, GRK2 expression levels and activity were elevated, accompanied by lowered β-AR density and signaling. Moreover, GRK2 inhibition by overexpression of the βARKct, the peptide inhibitor of GRK2, or cardiac specific GRK2 gene ablation, improved cardiac function and survival with the increases in β-AR density and β-AR responses in several HF models. These results suggest that GRK2 has a strong relationship with HF, and inhibition of GRK2 is a promising mechanism for the treatment of HF (OKAWA, T., et al., J. Med. Chem., 2017, pp 6942-6990, Vol. 60).

G protein-coupled receptor kinase 2 (GRK2) is emerging as a pivotal signalling hub able to integrate different transduction cascades. This ability appears to underlie its central role in different physiological and pathological conditions. Key mediators of cardiovascular function (such as catecholamines or angiotensin II) and components of the systemic milieu altered in insulin resistance conditions converge in increasing GRK2 levels in diverse cardiovascular cell types. In turn, GRK2 would simultaneously modulate several cardiovascular regulatory pathways, including GPCR and insulin signalling cascades, NO bioavailability and mitochondrial function. This fact can help explain the contribution of increased GRK2 levels to maladaptive cardiovascular function and remodeling. It also unveils GRK2 as a link between cardiovascular pathologies and co-morbidities such as obesity or type 2 diabetes. On the other hand, enhanced GRK2 expression, as observed in adipose tissues, liver or skeletal muscle during insulin resistance-related pathologies, could modify the orchestration of GPCR and insulin signalling in these crucial metabolic organs, and contribute to key features of the obese and insulin-resistant phenotype (MAYOR, Jr., F., et al., Cellular Signalling, 2018, pp 25-32, Vol. 41)

There remains a need for GRK2 inhibitor compounds that have pharmacokinetic and pharmacodynamic properties suitable for use as human pharmaceuticals for the treatment of for example, cardiac failure, cardiac hypertrophy, hypertension, Type II diabetes Mellitus, NASH, NAFLD, N-stage chronic kidney disease, kidney failure, etc.

### SUMMARY OF THE INVENTIONS

The present invention is directed to compounds of formula (I)

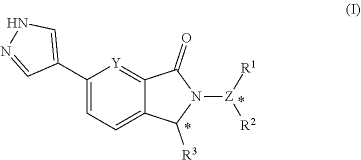

wherein

R¹ is phenyl; wherein the phenyl is optionally substituted with one to two substituents independently selected from the group consisting of halogen, hydroxy, C₁₋₂ alkyl and C₁₋₂ alkoxy;

Z is selected from the group consisting of CH and N;

R² and R³ are taken together with the atoms to which they are bound to form a fused ring structure selected from the group consisting of 1λ²-azepanyl, 2,3,6,7-tetrahydro-1λ²-azepinyl, 1,4λ²-oxazonanyl, (Z)-2,3,6,9-tetrahydro-5H-1,4λ²-oxazoninyl, 1λ²,2λ²-diazepanyl and 4,7-dihydro-3H-1λ²,2λ²-diazepinyl;

Y is selected from the group consisting of CH and N;

and pharmaceutically acceptable salts thereof.

The present invention is further directed to processes for the preparation of the compounds of formula (I). The present invention is further directed to a compound of formula (I) prepared according to any of the process(es) described herein.

Illustrative of the invention are pharmaceutical compositions comprising a pharmaceutically acceptable carrier and a compound of formula (I) as described herein. An illustration of the invention is a pharmaceutical composition made by mixing a compound of formula (I) as described herein and a pharmaceutically acceptable carrier. Illustrating the invention is a process for making a pharmaceutical composition comprising mixing a compound of formula (I) as described herein and a pharmaceutically acceptable carrier.

Exemplifying the invention are methods of treating a disease, disorder, or condition mediated by GRK2 activity as described herein, comprising administering to a subject in need thereof a therapeutically effective amount of any of the compounds or pharmaceutical compositions described above.

Exemplifying the invention are methods of treating a disease, disorder, or condition mediated by GRK2 activity such as obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD) and renal disorders (including, but not limited to n-stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure, a measured GFR equal or greater than 125 mL/min/1.73 m² (for example, a measured GFR equal or greater than 140 mL/min/1.73 m²)), comprising administering to a subject in need thereof a therapeutically effective amount of any of the compounds or pharmaceutical compositions described above.

In an embodiment, the present invention is directed to a compound of formula (I) for use as a medicament. In another embodiment, the present invention is directed to a compound of formula (I) for use in the treatment of a disorder mediated GRK2 activity such as obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD), n-stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m². In another embodiment, the present invention is directed to a composition comprising a compound of formula (I) for the treatment of a disorder mediated by GRK2 activity such as obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD), n-stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m².

Another example of the invention is the use of any of the compounds described herein in the preparation of a medicament for treating: (a) obesity, (b) excess weight, (c) impaired glucose tolerance (IGT), (d) impaired fasting glucose (IFT), (e) gestational diabetes, (f) Type II diabetes mellitus, (g) Syndrome X (also known as Metabolic Syndrome), (h) nephropathy, (i) neuropathy, (j) retinopathy, in a subject in need thereof.

Another example of the invention is the use of any of the compounds described herein in the preparation of a medicament for treating: (a) cardiac failure, (b) cardiac hypertrophy, (c) cardiac fibrosis, (d) hypertension, (e) angina, (f) atherosclerosis, (g) heart disease, (h) heart attack, (i) ischemia, (j) stroke, (k) nerve damage or poor blood flow in the feet and (l) sepsis-associated encephalopathy (SAE), in a subject in need thereof.

Another example of the invention is the use of any of the compounds described herein in the preparation of a medicament for treating: (a) non-alcoholic steatohepatitis (NASH) and (b) non-alcoholic fatty liver disease (NAFLD), in a subject in need thereof.

Another example of the invention is the use of any of the compounds described herein in the preparation of a medicament for treating a disorder as described herein. Another example of the invention is the use of any of the compounds described herein in the preparation of a medicament for treating: (a) n-stage chronic kidney disease, (b) chronic kidney disease, (c) acute renal failure, (d) nephrotic syndrome, (e) renal hyperfiltrative injury, (f) hyperfiltrative diabetic nephropathy, (g) renal hyperfiltration, (h) glomerular hyperfiltration, (i) renal allograft hyperfiltration, (j) compensatory hyperfiltration, (k) hyperfiltrative chronic kidney disease, (l) hyperfiltrative acute renal failure and (m) a measured GFR equal or greater than 125 mL/min/1.73 m², in a subject in need thereof.

In another example, the present invention is directed to a compound as described herein, for use in a method for treating a disorder as described herein. In another example, the present invention is directed to a compound as described herein, for use in a methods for treating a disorder selected from the group consisting of obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD), n-stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m², in a subject in need thereof.

### DETAILED DESCRIPTION OF THE INVENTION

The present invention is directed to compounds of formula (I)

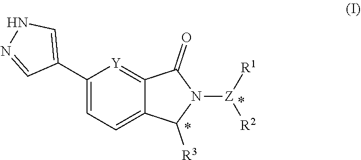

wherein Z, R¹, R², R³ and Y are as herein defined; and isotopologues and pharmaceutically acceptable salts thereof. In the compounds of formula (I), when Z is CH, a stereocenter exists at the Z position, as denoted by the “*” in the structures herein. In the compounds of formula (I), when Z is N, no stereocenter exists at the Z position. In the compounds of formula (I) a stereocenter is present at the carbon atom bound to R³, as denoted by the “*” in the structures herein.

The compounds of the present invention are useful in the treatment of diseases, disorders and complications associated with GRK2 activity selected from the group consisting of obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD) and renal disorders (including, but not limited to n-stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure, a measured GFR equal or greater than 125 mL/min/1.73 m² (for example, a measured GFR equal or greater than 140 mL/min/1.73 m²)).

In an embodiment, the compounds of the present invention are useful in the treatment of diseases, disorders and complications associated with GRK2 activity selected from the group consisting of (a) obesity, (b) excess weight, (c) impaired glucose tolerance (IGT), (d) impaired fasting glucose (IFT), (e) gestational diabetes, (f) Type II diabetes mellitus, (g) Syndrome X (also known as Metabolic Syndrome), (h) nephropathy, (i) neuropathy, (j) retinopathy, (k) cardiac failure, (l) cardiac hypertrophy, (m) cardiac fibrosis, (n) hypertension, (o) angina, (p) atherosclerosis, (q) heart disease, (r) heart attack, (s) ischemia, (t) stroke, (u) nerve damage or poor blood flow in the feet, (v) sepsis-associated encephalopathy (SAE), (w) non-alcoholic steatohepatitis (NASH), (x) non-alcoholic fatty liver disease (NAFLD) (y) n-stage chronic kidney disease, (z) chronic kidney disease, (aa) acute renal failure, (ab) nephrotic syndrome, (ac) renal hyperfiltrative injury, (ad) hyperfiltrative diabetic nephropathy, (ae) renal hyperfiltration, (af) glomerular hyperfiltration, (ag) renal allograft hyperfiltration, (ah) compensatory hyperfiltration, (ai) hyperfiltrative chronic kidney disease, (aj) hyperfiltrative acute renal failure and (ak) a measured GFR equal or greater than 125 mL/min/1.73 m².

In an embodiment, the compounds of the present invention are useful in the treatment of diseases, disorders and complications associated with GRK2 activity selected from the group consisting of obesity, excess weight, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy, diabetic retinopathy, cardiac failure, cardiac hypertrophy, hypertension, angina, atherosclerosis, non-alcoholic steatohepatitis (NASH), non-alcoholic fatty liver disease (NAFLD), n-stage chronic kidney disease, chronic kidney disease, acute renal failure, and a measured GFR equal or greater than 125 mL/min/1.73 m².

In another embodiment, the compounds of the present invention are useful in the treatment of diseases, disorders and complications associated with GRK2 activity selected from the group consisting of obesity, excess weight, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy, diabetic retinopathy, non-alcoholic steatohepatitis (NASH), non-alcoholic fatty liver disease (NAFLD), n-stage chronic kidney disease, chronic kidney disease, acute renal failure, and a measured GFR equal or greater than 125 mL/min/1.73 m².

In an embodiment, the compounds of the present invention are useful in the treatment of diseases, disorders and complications associated with GRK2 activity selected from the group consisting of obesity, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy and diabetic retinopathy.

In another embodiment, the compounds of the present invention are useful in the treatment of diseases, disorders and complications associated with GRK2 activity selected from the group consisting of cardiac failure, cardiac hypertrophy, hypertension and atherosclerosis.

In another embodiment, the compounds of the present invention are useful in the treatment of diseases, disorders and complications associated with GRK2 activity selected from the group consisting of non-alcoholic steatohepatitis (NASH) and non-alcoholic fatty liver disease (NAFLD).

In another embodiment, the compounds of the present invention are useful in the treatment of renal diseases, disorders and complications associated with GRK2 activity selected from the group consisting of n-stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m² (for example, a measured GFR equal or greater than 140 mL/min/1.73 m²)).

In an embodiment, the present invention is directed to compounds of formula (I) wherein Z is CH. In an embodiment, the present invention is directed to compounds of formula (I) wherein Z is N.

In an embodiment, the present invention is directed to compounds of formula (I) wherein Y is CH. In an embodiment, the present invention is directed to compounds of formula (I) wherein Y is N.

In an embodiment, the present invention is directed to compounds of formula (I) wherein R¹ is phenyl; wherein the phenyl is optionally substituted with one to two substituents independently selected from the group consisting of fluoro, hydroxy and methoxy.

In an embodiment, the present invention is directed to compounds of formula (I) wherein R¹ is selected from the group consisting of R*-(3-hydroxy-phenyl), S*-(3-hydroxy-phenyl), 3-methoxyphenyl, R-(3-methoxyphenyl), S-(3-methoxyphenyl), R*-(3-methoxyphenyl), S*-(3-methoxyphenyl), R*-(3-methoxy-5-fluoro-phenyl) and S*-(3-methoxy-5-fluoro-phenyl). In another embodiment, the present invention is directed to compounds of formula (I) wherein R¹ is selected from the group consisting R*-(3-hydroxy-phenyl), S*-(3-hydroxy-phenyl), R-(3-methoxy-phenyl), R*-(3-methoxy-phenyl), S*-(3-methoxy-phenyl), R*-(3-methoxy-5-fluoro-phenyl) and S*-(3-methoxy-5-fluoro-phenyl). In another embodiment, the present invention is directed to compounds of formula (I) wherein R¹ is S-(3-methoxyphenyl).

In another embodiment, the present invention is directed to compounds of formula (I) wherein R¹ is selected from the group consisting 3-hydroxyphenyl, 3-methoxyphenyl, 3-methoxy-5-fluoro-phenyl and enantiomers thereof. In another embodiment, the present invention is directed to compounds of formula (I) wherein R¹ is selected from the group consisting 3-methoxyphenyl, 3-methoxy-5-fluoro-phenyl and enantiomers thereof. In another embodiment, the present invention is directed to compounds of formula (I) wherein R¹ is 3-methoxyphenyl and enantiomers thereof.

In an embodiment, the present invention is directed to compounds of formula (I) wherein R² and R³ are taken together with the carbon atoms to which they are bound to form 1λ²-azepanyl, compounds of the formula (Ia)

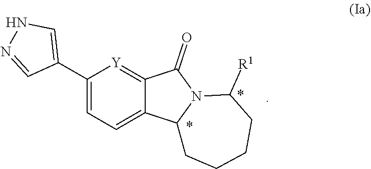

In an embodiment, the present invention is directed to compounds of formula (I) wherein R² and R³ are taken together with the carbon atoms to which they are bound to form 2,3,6,7-tetrahydro-1λ²-azepinyl, compounds of formula (Ib)

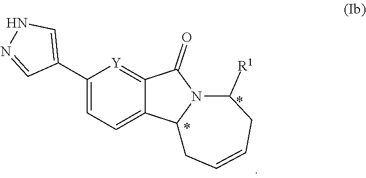

In an embodiment, the present invention is directed to compounds of formula (I) wherein R² and R³ are taken together with the carbon atoms to which they are bound to form 1,4λ²-oxazonanyl, i.e. a compound of the formula (Ic)

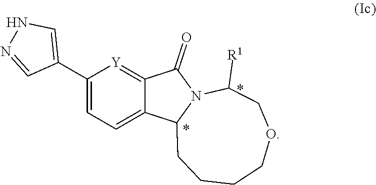

In an embodiment, the present invention is directed to compounds of formula (I) wherein R² and R³ are taken together with the carbon atoms to which they are bound to form (Z)-2,3,6,9-tetrahydro-5H-1,4λ²-oxazoninyl, i.e. a compound of the formula (Id)

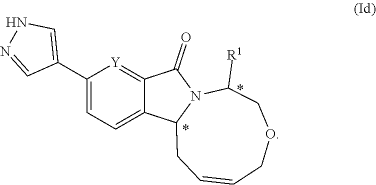

In an embodiment, the present invention is directed to compounds of formula (I) wherein R² and R³ are taken together with the carbon atoms to which they are bound to form 1λ²,2λ²-diazepanyl, i.e. a compound of the formula (Ie).

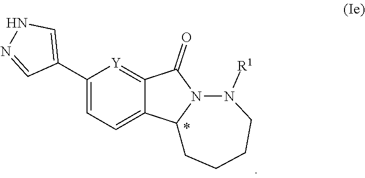

In an embodiment, the present invention is directed to compounds of formula (I) wherein R² and R³ are taken together with the carbon atoms to which they are bound to form 4,7-dihydro-3H-1λ²,2λ²-diazepinyl, i.e. a compound of the formula (If).

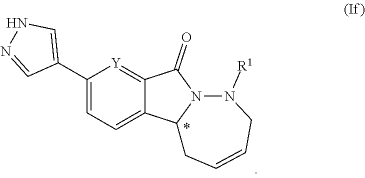

In an embodiment, the present invention is directed to a compound of formula (I) selected from the group consisting of

(7R*)-7-(3-methoxyphenyl)-3-(1H-pyrazol-4-yl)-7,8,9,10,11,11a-hexahydro-5H-azepino[2,1-a]isoindol-5-one;

(Rac)-1-(3-methoxyphenyl)-8-(1H-pyrazol-4-yl)-1,2,3,4,5,5a-hexahydro-10H-[1,2]diazepino[7,1-a]isoindol-10-one;

(7S*)-7-(3-hydroxyphenyl)-3-(1H-pyrazol-4-yl)-7,8,9,10,11,11a-hexahydro-5H-azepino[2,1-a]isoindol-5-one;

and pharmaceutically acceptable salts thereof.

Additional embodiments of the present invention, include those wherein the substituents selected for one or more of the variables defined herein (i.e. Z, R¹, R², R³, Y, etc.) are independently selected to be any individual substituent or any subset of substituents selected from the complete list as defined herein. Additional embodiments of the present invention, include those wherein the substituents selected for one or more of the variables defined herein (i.e. Z, R¹, R², R³, Y, etc.) are independently selected to correspond to any of the embodiments as defined herein.

In another embodiment of the present invention is any single compound or subset of compounds selected from the representative compounds listed in Table 1, below.

Representative compounds of the present invention are as listed in Table 1, below. Wherein the compound listed in Table 1 below, Z is CH, such that a stereocenter exists that the Z position, the stereo-configuration at said stereo-center is defined in the listing of the substituents in the column headed “R¹”. Where a stereogenic center is present and an S* or R* designation is noted, the S* and R* designations indicate that the compound was prepared in an enantiomeric excess of one of the stereo-isomers, although the exact stereo-configuration of the center was not determined. Where a stereogenic center is present and an S or R designation is noted, the S and R designations indicate that the compound was prepared in an enantiomeric excess of one of the stereo-isomers, and further that the exact stereo-configuration of the center was determined to be S or R, as noted.

TABLE 1
Representative Compounds of Formula (I)

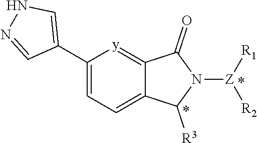

| | | | | | R² + R³ together with |
|---|---|---|---|---|---|
| | | | | R³- | atoms to which they |
| ID No. | R¹ | Z | Y | Stereo | are bound |
| 1 | R*-(3-methoxy-phenyl) | CH | CH | rac | 1λ²-azepanyl |
| 2 | S*-(3-hydroxy-phenyl) | CH | CH | rac | 1λ²-azepanyl |
| 3 | S*-(3-methoxy-phenyl) | CH | CH | rac | 1λ²-azepanyl |
| 4 | R*-(3-methoxy-5- | CH | CH | rac | 1λ²-azepanyl |
| | fluoro-phenyl) | | | | |
| 5 | R-(3-methoxy-phenyl) | CH | CH | S* | 1λ²-azepanyl |
| 6 | R*-(3-hydroxy-phenyl) | CH | CH | rac | 1λ²-azepanyl |
| 7 | S*-(3-methoxy-5- | CH | CH | rac | 1λ²-azepanyl |
| | fluoro-phenyl) | | | | |
| 8 | S-(3-methoxy-phenyl) | CH | CH | rac | (Z)-2,3,6,9-tetrahydro- |
| | | | | | 5H-1,4λ²-oxazoninyl |
| 9 | S-(3-methoxy-phenyl) | CH | CH | rac | 1,4λ²-oxazonanyl |
| 12 | S*-(3-methoxy-phenyl) | CH | N | rac | 1λ²-azepanyl |
| 13 | R*-(3-methoxy-phenyl) | CH | N | rac | 1λ²-azepanyl |
| 14 | S-(3-methoxy-phenyl) | CH | N | rac | (Z)-2,3,6,9-tetrahydro- |
| | | | | | 5H-1,4λ²-oxazoninyl |
| 15 | 3-methoxy-phenyl | N | CH | rac | 1λ², 2λ²-diazepanyl |
| 16 | 3-methoxy-phenyl | N | CH | rac | 4,7-dihydro-3H-1λ², |
| | | | | | 2λ²-diazepinyl |

### Definitions

As used herein, “halogen” shall mean chloro, bromo, fluoro and iodo, preferably chloro, bromo or fluoro, more preferably fluoro.

As used herein, the term “C_{X-Y}alkyl” wherein X and Y are integers, whether used alone or as part of a substituent group, include straight and branched chains containing between X and Y carbon atoms. For example, alkyl radicals include straight and branched chains of between 1 and 4 carbon atoms, including methyl, ethyl, propyl, isopropyl, butyl, isobutyl, sec-butyl and t-butyl.

As used herein, unless otherwise noted, “C₁₋₄alkoxy” shall denote an oxygen ether radical of the above described straight or branched chain alkyl groups containing one to four carbon atoms. For example, methoxy, ethoxy, n-propoxy, isopropoxy, sec-butoxy, t-butoxy, and the like.

When a particular group is “substituted” (e.g. C_{X-Y}alkyl, C_{X-Y}alkoxy, C_{X-Y}cycloalkyl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

As used herein, the notation “*” shall denote the presence of a stereogenic center.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

As used herein, unless otherwise noted, the term “isotopologues” shall mean molecules that differ only in their isotopic composition. More particularly, an isotopologue of a molecule differs from the parent molecule in that it contains at least one atom which is an isotope (i.e. has a different number of neutrons from its parent atom).

For example, isotopologues of water include, but are not limited to, “light water” (HOH or H₂O), “semi-heavy water” with the deuterium isotope in equal proportion to protium (HDO or ¹H²HO), “heavy water” with two deuterium isotopes of hydrogen per molecule (D₂O or ²H₂O), “super-heavy water” or tritiated water (T₂O or ³H₂O), where the hydrogen atoms are replaced with tritium (³H) isotopes, two heavy-oxygen water isotopologues (H₂¹⁸O and H₂¹⁷O) and isotopologues where the hydrogen and oxygen atoms may each independently be replaced by isotopes, for example the doubly labeled water isotopologue D₂¹⁸O.

It is intended that within the scope of the present invention, any one or more element(s), in particular when mentioned in relation to a compound of formula (I), shall comprise all isotopes and isotopic mixtures of said element(s), either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. Radiolabelled compounds of formula (I) may comprise one or more radioactive isotope(s) selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

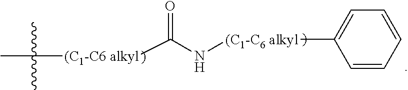

Abbreviations used in the specification, particularly the Schemes and Examples, are as listed in the Table A, below:

TABLE A
Abbreviations

| | |
|---|---|
| AcOEt or EA or EtOAc = | Ethyl Acetate |
| ACN or MeCN = | Acetonitrile |
| ATP = | Adenosine Triphosphate |
| BF₃ · Et₂O = | Boron trifluoride diethyl etherate |
| Boc or BOC = | tert-Butoxycarbonyl |
| t-BuOK or KO-t-Bu = | Potassium tert-Butoxide |
| Cu(OAc)₂ = | Copper (II) Acetate |
| DCM = | Dichloromethane |
| DIPEA or DIEA = | Diisopropylethylamine |
| DMF = | N,N-Dimethylformamide |
| DMSO = | Dimethylsulfoxide |
| EA or EtOAc = | Ethyl Acetate |
| ES = | ElectroSpray (mass spectroscopy) |
| Et₂O = | Diethyl Ether |
| Et₃N or TEA = | Triethylamine |
| EtOAc or EA = | Ethyl acetate |
| FBS = | Fetal Bovine Serum |
| GFR = | Glomular Filtration Rate |
| GRK2 = | G protein-coupled Receptor Kinase 2 |
| HEPES = | 4-(2-Hydroxyethyl)-1-Piperizine |
| | EthaneSulfonic Acid |
| HPLC = | High Pressure Liquid Chromatography |
| IFG = | Impaired fasting glucose |
| IGT = | Impaired glucose tolerance |
| KO-t-Bu = | Potassium tert-Butoxide |
| LC-MS or LC/MS = | Liquid chromatography-mass spectrometry |
| LiHMDS = | Lithium bis(trimethylsilyl)amide |
| MeCN or I = | Acetonitrile |
| MeOH = | Methanol |
| mesylate or OMs = | Methanesulfonate (i.e. —O—SO₂—CH₃) |
| MOM = | Methoxy methyl |
| MS (Å) = | Molecular Sieves (Angstroms) |
| NaHMDS = | Sodium bis(trimethylsilyl)amide |
| NAFLD = | Non-alcoholic fatty liver disease |
| NASH = | Non-alcoholic steatohepatitis, |
| NMR = | Nuclear Magnetic Resonance |
| OGTT = | Oral Glucose Tolerance Test |
| OMs or mesylate = | Methanesulfonate (i.e. —O—SO₂—CH₃) |
| OTf or triflate = | Trifluoromethanesulfonyl |
| | (i.e. —O—SO₂ = —CF₃) |
| OTfCl = | Trifluoromethanesulfonyl chloride |
| OTs or tosylate = | p-Toluenesulfonate ) |
| | (i.e. —O—SO₂—(p-methylphenyl) |
| Pd/C = | Palladium on Carbon (catalyst) |
| PdCl₂dppf = | [1,1′-Bis(diphenylphosphino)ferrocene] |
| | Palladium (II) Dichloride |
| PdCl₂(PPh₃)₂ = | Bis(triphenylphosphine) |
| | Pallasdium (II) Dichloride |
| Pd(PPh₃)₄ = | Tetrakistriphenylphosphine palladium (0) |
| PE = | Petroleum ether |
| PPh₃ = | Triphenylphosphine |
| I-PrOH = | Isopropanol |
| Rac or rac = | Racemic |
| TFA = | Trifluoroacetic Acid |
| THF = | Tetrahydrofuran |
| THP = | Tetrahydropyran |
| Ti(OPr-1)₄ = | Titanium (IV) lsopropoxide |
| TLC = | Thin Layer Chromatography |
| TMS = | Trimethylsilyl |
| Tosylate or OTs = | p-Toluenesulfonate |
| | (i.e. —O—SO₂—(p-methylphenyl)) |

As used herein, unless otherwise noted, the term “isolated form” shall mean that the compound is present in a form which is separate from any solid mixture with another compound(s), solvent system or biological environment. In an embodiment of the present invention, the compound of formula (I) is present in an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I) is present as a substantially pure form.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In an embodiment of the present invention, the compound of formula (I) is present in a form which is substantially free of corresponding salt form(s).

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, slow the progression of the disease or disorder, or eliminate the disease, condition, or disorder.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems.

One skilled in the art will recognize that wherein a reaction step of the present invention may be carried out in a variety of solvents or solvent systems, said reaction step may also be carried out in a mixture of the suitable solvents or solvent systems.

One skilled in the art will further recognize that the reaction or process step(s) as herein described are allowed to proceed for a sufficient period of time until the reaction is complete, as determined by any method known to one skilled in the art, for example, chromatography (e.g. HPLC). In this context a “completed reaction or process step” shall mean that the reaction mixture contains a significantly diminished amount of the starting material(s)/reagent(s) and a significantly reduced amount of the desired product(s), as compared to the amounts of each present at the beginning of the reaction.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed descriptions which follow herein. One skilled in the art will recognize that the listing of said examples is not intended, and should not be construed, as limiting in any way the invention set forth in the claims which follow thereafter.

As used herein, unless otherwise noted, the term “aprotic solvent” shall mean any solvent that does not yield a proton. Suitable examples include, but are not limited to DMF, 1,4-dioxane, THF, acetonitrile, pyridine, dichloroethane, dichloromethane, MTBE, toluene, acetone, and the like.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein, unless otherwise noted, the term “oxygen protecting group” shall mean a group which may be attached to an oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be found in texts such as T. W. Greene & P. G. M. Wuts, Protective Groups in Organic Synthesis, John Wiley & Sons, 1991.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### General Synthesis Schemes

Compounds of formula (I) of the present invention may be synthesized according to the general synthesis schemes described below. The preparation of the various starting materials used in the synthesis schemes which follow hereinafter is well within the skill of persons versed in the art.

Compounds of formula (I) wherein R² and R³ are taken together with the atoms to which they are bound to form 1λ²-azepanyl or 2,3,6,7-tetrahydro-1λ2-azepinyl may be prepared as described in Scheme 1, below.

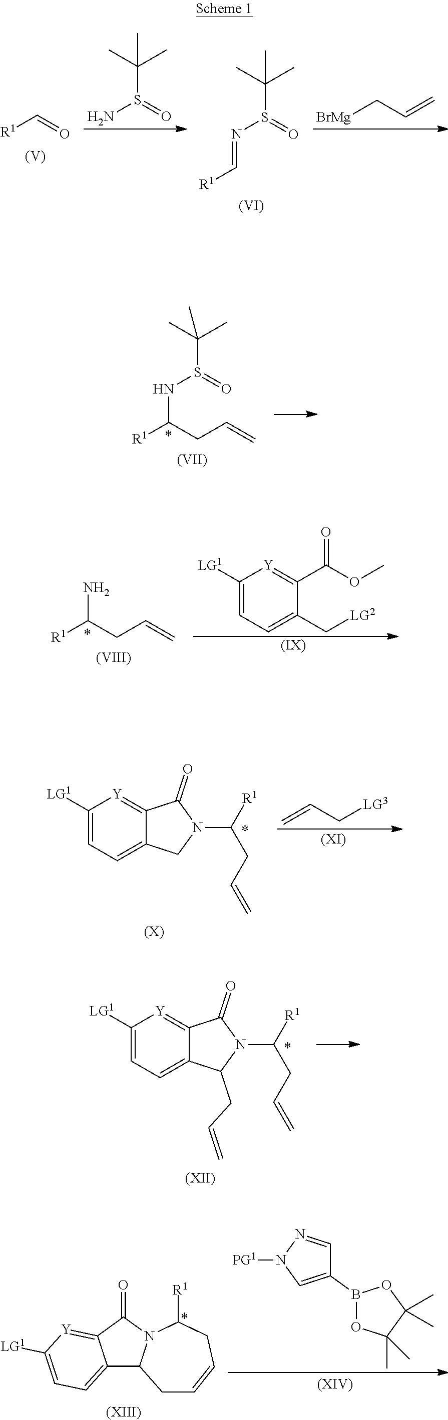

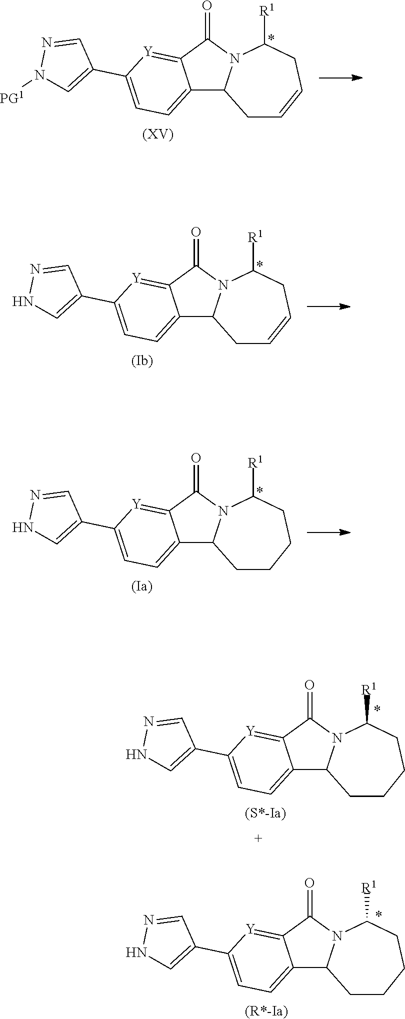

Accordingly, a suitably substituted aldehyde of formula (V), a known compound or compound prepared by known methods, is reacted with 2-methylpropane-2-sulfinamide, a known compound; in the presence of a suitably selected Lewis acid/dehydrating agents such as Ti(OCH₂CH₃)₄, Ti(OPr-i)₄, CuSO₄, MgSO₄, and the like; in a suitably selected solvent such as THF, DCM, and the like; at a temperature in the range of from about 0° C. to about reflux condition, for example at about 60° C.; to yield the corresponding compound of formula (VI).

The compound of formula (VI) is reacted with allylmagnesium bromide, a known compound; in a suitably selected anhydrous solvent such as THF, DCM, Et₂O, and the like; at about room temperature; to yield the corresponding compound of formula (VII).

The compound of formula (VII) is reacted with a suitably selected acid such as HCl, NH₄Cl, and the like; in a suitably selected solvent such as methanol, Et₂O, 1,4-dioxane, water, and the like; at about room temperature; to yield the corresponding compound of formula (VIII).

The compound of formula (VIII) is reacted with a suitably substituted compound of formula (IX) wherein LG¹ is a suitably selected leaving group such as Br, I, OTf, Cl, and the like, wherein LG² is a suitably selected leaving group such as Br, OMs, Cl, and the like, and wherein LG¹ and LG² may be the same or different, and are selected such that LG¹ remains intact; in the presence of a suitably selected organic base such as DIPEA, Et₃N, pyridine, and the like; in a suitably selected solvent such as acetonitrile, DMF, THF, and the like; to yield the corresponding compound of formula (X).

The compound of formula (X) is reacted with a suitably substituted compound of formula (XI), wherein LG³ is a suitably selected leaving group such as Br, I, OMs, and the like, a known compound or compound prepared by known methods; in the presence of a suitably selected base such as NaH, LiHMDS, NaHMDS, and the like; in a suitably selected solvent such as THF, Et₂O, DMF, and the like; at a temperature in the range of from about 0° C. to about 80° C., for example at about 75° C.; to yield the corresponding compound of formula (XII).

The compound of formula (XII) is reacted to effect ring closure, reacting with for example first and second-generation Grubb's reagents, Schrock catalyst, Hoveyda-Grubbs catalyst, and the like; in a suitably selected solvent such as DCM, toluene, EtOAc, and the like; at about room temperature; to yield the corresponding compound of formula (XIII).

The compound of formula (XIII) is reacted with a suitably substituted compound of formula (XIV), wherein PG¹ is a suitably selected nitrogen protecting group such as Boc, and the like, a known compound or compound prepared by known methods; in the presence of a suitably selected coupling agent such as Pd(PPh₃)₄, PdCl₂dppf, PdCl₂(PPh₃)₂, and the like; in the presence of a suitably selected base such as K₂CO₃, Na₂CO₃, K₃PO₄, and the like; in the presence of a suitably selected in a suitably selected solvent such as a mixture of 1,4-dioxane and water, toluene, DMF, and the like; at a temperature in the range of from about 60° C. to about 110° C., for example at about 85° C.; to yield the corresponding compound of formula (XV).

The compound of formula (XV) is de-protected according to known methods, to yield the corresponding compound of formula (Ib). One skilled in the art will recognize that wherein PG¹ is BOC, the BOC protecting group is removed under TFA or aqueous NH₄Cl work up.

One skilled in the art will recognize that the compound of formula (Ib) may be separated into its corresponding S* and R* enantiomers according to known methods, for example by direct chiral resolution or separation on a chiral HPLC column.

The compound of formula (Ib) is reacted with hydrogen gas; in the presence of a suitably selected catalyst such as Pd/C; in a suitably selected solvent such as methanol, EtOAc, acetonitrile, and the like; at about room temperature; to yield the corresponding compound of formula (Ia).

One skilled in the art will recognize that the compound of formula (Ia) may be separated into its corresponding S* and R* enantiomers according to known methods, for example by direct chiral resolution or separation on a chiral HPLC column.

Compounds of formula (I) wherein R² and R³ are taken together with the atoms to which they are bound to form 1,4λ²-oxazonanyl or (Z)-2,3,6,9-tetrahydro-5H-1,4λ²-oxazoninyl, may be prepared as described in Scheme 2, below.

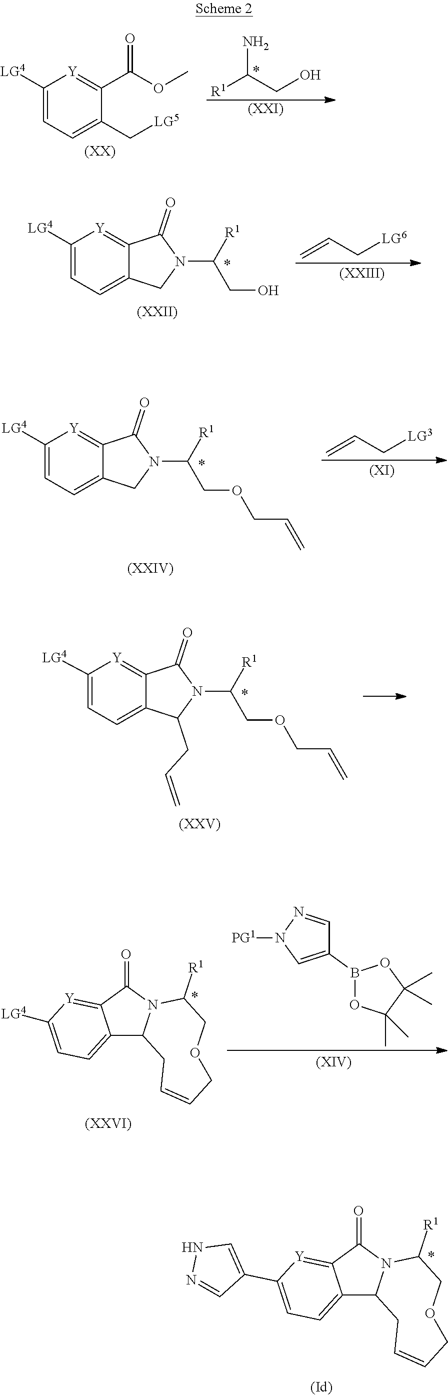

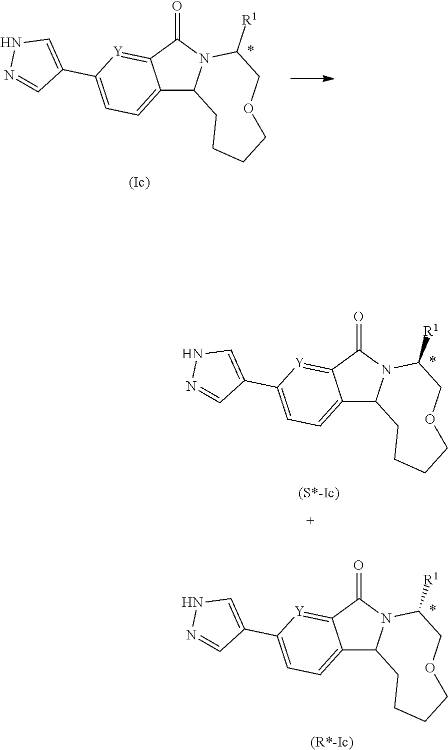

Accordingly, a suitably substituted compound of formula (XX), wherein LG⁴ is a suitably selected leaving group such as Br, I, OTf, and the like, and wherein LG⁵ is a suitably selected leaving group such as Br, Cl, OTs, and the like, and wherein LG⁴ and LG⁵ may be the same or different, and are selected such that LG⁴ remains intact, a known compound or compound prepared by known methods is reacted with a suitably substituted compound of formula (XXI), wherein the compound or formula (XXI) is optionally present in an enantiomeric excess of the corresponding R* or S* enantiomer, a known compound or compound prepared by known methods; in the presence of a suitably selected organic base such as DIPEA, Et₃N, Cs₂CO₃, and the like; in a suitably selected solvent such as acetonitrile, DMF, 1,4-dioxane, and the like; to yield the corresponding compound of formula (XXII).

The compound of formula (XXII) is reacted with a suitably substituted compound of formula (XXIII), wherein LG⁶ is a suitably selected leaving group such as I, Br, OMs, and the like, a known compound or compound prepared by known methods; in the presence of a suitably selected coupling agent such as Ag₂O, NaH, KO-t-Bu, and the like; in a suitably selected solvent such as acetonitrile, THF, DMF, and the like; at about room temperature; to yield the corresponding compound of formula (XXIV).

The compound of formula (XXIV) is reacted with a suitably substituted compound of formula (XI), wherein LG³ is a suitably selected leaving group such as Br, I, OMs, and the like, a known compound or compound prepared by known methods; in the presence of a suitably selected base such as NaH, LiHMDS, NaHMDS, and the like; in a suitably selected solvent such as THF, Et₂O, DMF, and the like; at a temperature in the range of from about 0° C. to about 100° C., for example at about 75° C.; to yield the corresponding compound of formula (XXV).

The compound of formula (XXV) is reacted to effect ring closure, reacting with for example Grubb's first and second-generation catalysts, Schrock catalyst, Hoveyda-Grubbs catalyst, and the like; in a suitably selected solvent such as DCM, toluene, EtOAc, and the like; at about room temperature; to yield the corresponding compound of formula (XXVI).

The compound of formula (XXVI) is reacted with a suitably substituted compound of formula (XIV), wherein PG¹ is a suitably selected nitrogen protecting group such as BOC, a known compound or compound prepared by known methods; in the presence of a suitably selected coupling agent such as Pd(PPh₃)₄, PdCl₂dppf, PdCl₂(PPh₃)₂, and the like; in the presence of a suitably selected base such as K₂CO₃, Na₂CO₃, K₃PO₄ and the like; in the presence of a suitably selected in a suitably selected solvent such as a mixture of 1,4-dioxane and water, toluene, DMF, and the like; at a temperature in the range of from about 60° C. to about 130° C., for example at about 85° C.; to yield the corresponding compound of formula (XXVII).

The compound of formula (XXVII) is de-protected according to known methods, to yield the corresponding compound of formula (Id). One skilled in the art will recognize that wherein PG¹ is BOC, the BOC protecting group is removed under TFA or aqueous NH₄Cl work-up.

One skilled in the art will recognize that the compound of formula (Id) may be separated into its corresponding S* and R* enantiomers according to known methods, for example by direct chiral resolution or separation on a chiral HPLC column.

The compound of formula (Id) is reacted with hydrogen gas; in the presence of a suitably selected catalyst such as Pd/C; in a suitably selected solvent such as methanol, i-PrOH, EtOAc, and the like; at about room temperature; to yield a mixture of the corresponding S* and R* enantiomers of the compound of formula (Ic).

One skilled in the art will recognize that the compound of formula (Ic) may be separated into its corresponding S* and R* enantiomers according to known methods, for example by direct chiral resolution or separation on a chiral HPLC column.

Compounds of formula (I) wherein R² and R³ are taken together with the atoms to which they are bound to form 1λ²,2λ²-diazepanyl or 4,7-dihydro-3H-1λ²,2λ²-diazepinyl may be prepared as described in Scheme 3, below.

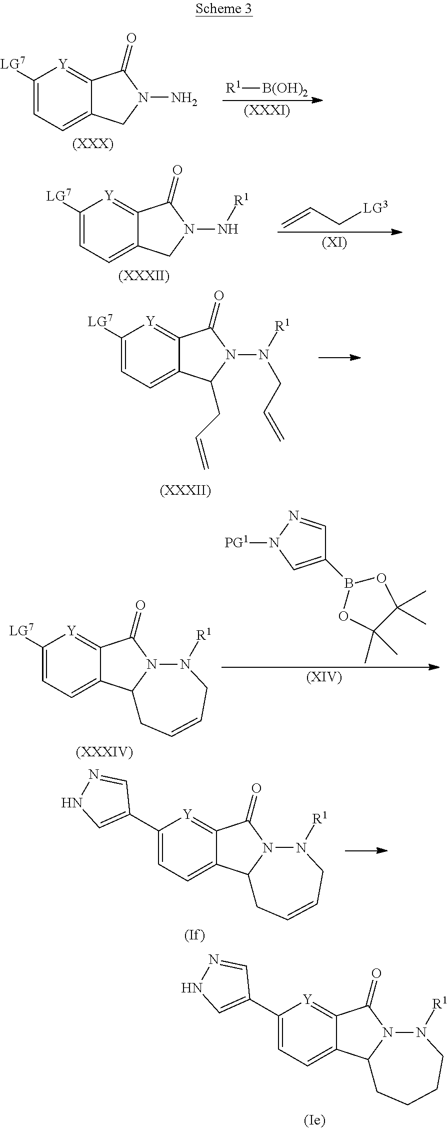

Accordingly, a suitably substituted compound of formula (XXX), wherein LG⁷ is a suitably selected leaving group such as Br, I, OTf, and the like, a known compound or compound prepared by known methods, is reacted with a suitably substituted compound of formula (XXXI), a known compound or compound prepared by known methods; in the presence of a suitably selected organic base such as TEA, DIPEA, pyridine, and the like; in the presence of anhydrous Cu(OAc)₂, and the like; in a suitably selected solvent such as DCM, MeCN, toluene, and the like; in the presence of molecular sieves; at about room temperature; to yield the corresponding compound of formula (XXXII).

The compound of formula (XXXII) is reacted with a compound of formula (XI), wherein LG³ is a suitably selected leaving group such as Br, I, OMs, and the like, a known compound or compound prepared by known methods; in the presence of a suitably selected base such as NaH, LiHMDS, NaHMDS, and the like; in a suitably selected solvent such as THF, Et₂O, DMF, and the like; at a temperature in the range of from about 0° C. to about 100° C., for example at about 75° C.; to yield the corresponding compound of formula (XXXIII).

The compound of formula (XXXIII) is reacted to effect ring closure, reacting with for example Grubb's first and second-generation catalysts, Schrock catalyst, Hovedya-Grubbs catalyst, and the like; in a suitably selected solvent such as DCM, toluene, EtOAc, and the like; at about room temperature; to yield the corresponding compound of formula (XXXIV).

The compound of formula (XXXIV) is reacted with a suitably substituted compound of formula (XIV), wherein PG¹ is a suitably selected nitrogen protecting group such as BOC, a known compound or compound prepared by known methods; in the presence of a suitably selected coupling agent such as Pd(PPh₃)₄, Pdcl₂ppf, Pdcl₃(PPh₃)₂, and the like; in the presence of a suitably selected base such as K₂CO₃, Na₂CO₃, K₃PO₄, and the like; in the presence of a suitably selected in a suitably selected solvent such as a mixture of 1,4-dioxane and water, toluene, DMF, and the like; at a temperature in the range of from about 60° C. to about 130° C., for example at about 85° C.; to yield the corresponding compound of formula (XXXV).

The compound of formula (XXXV) is de-protected according to known methods, to yield the corresponding compound of formula (If), as a mixture of the corresponding S* and R* enantiomers. One skilled in the art will recognize that wherein PG¹ is BOC, the BOC protecting group is removed under TFA or aqueous NH₄Cl work-up.

One skilled in the art will recognize that the compound of formula (If) may be separated into its corresponding S* and R* enantiomers according to known methods, for example by direct chiral resolution or separation on a chiral HPLC column.

The compound of formula (If) is reacted with hydrogen gas; in the presence of a suitably selected catalyst such as Pd/C; in a suitably selected solvent such as methanol, MeCN, EtOAc, and the like; at about room temperature; to yield a mixture of the corresponding S* and R* enantiomers of the compound of formula (Ie).

One skilled in the art will recognize that the compound of formula (Ie) may be separated into its corresponding S* and R* enantiomers according to known methods, for example by direct chiral resolution or separation on a chiral HPLC column.

One skilled in the art will recognize that compounds of formula (I) wherein R¹ is phenyl substituted with one to two hydroxy groups, may be prepared from the corresponding compound of formula (I) wherein the phenyl positions to be substituted with hydroxy group(s) are instead substituted with methoxy group(s), by reacting with a suitably selected demethylating reagent such as BBr₃, AlCl₃/NaI, BF₃.Et₂O/NaI, and the like; in a suitably selected solvent such as DCM, MeCN, ClCH₂CH₂Cl, and the like; according to known methods.

### Pharmaceutical Compositions

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus, for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg to about 1000 mg or any amount or range therein, and may be given at a dosage of from about 0.05 mg/day to about 300 mg/day, or any amount or range therein, preferably from about 0.1 mg/day to about 100 mg/day, or any amount or range therein, preferably from about 1 mg/day to about 50 mg/day, or any amount or range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.01 mg to about 1,000 mg, or any amount or range therein, of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

Carriers that may be used in the present invention include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methylcellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of disorders mediated by GRK2 activity, is required.

The daily dosage of the products may be varied over a wide range from about 0.01 mg to about 1,000 mg per adult human per day, or any amount or range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug may be ordinarily supplied at a dosage level of from about 0.005 mg/kg to about 10 mg/kg of body weight per day, or any amount or range therein. Preferably, the range is from about 0.01 to about 5.0 mg/kg of body weight per day, or any amount or range therein, more preferably, from about 0.1 to about 1.0 mg/kg of body weight per day, or any amount or range therein, more preferably, from about 0.1 to about 0.5 mg/kg of body weight per day, or any amount or range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1: Compound #10

### (1S,Z)-1-(3-methoxyphenyl)-10-(1H-pyrazol-4-yl)-1,2,7,7a-tetrahydro-[1,4]oxazonino[5,4-a]isoindol-12(4H)-one

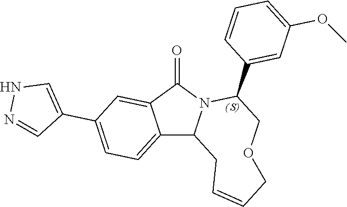

### Step A: (S)-6-bromo-2-(2-hydroxy-1-(3-methoxyphenyl)ethyl)isoindolin-1-one

2-Amino-2-(3-methoxyphenyl)ethanol (1.5 g, 8.971 mmol, 1 equiv) was dissolved in acetonitrile (15 ml), then N,N-diisopropylethylamine (4.448 mL, 26.913 mmol, 3 equiv), methyl 5-bromo-2-(bromomethyl)benzoate (3.039 g, 9.868 mmol, 1.1 equiv) were added and stirred overnight at 85° C. The resulting mixture was concentrated and the residue was applied onto a silica gel column with EtOAc/petroleum ether (0-15%), to yield (S)-6-bromo-2-(2-hydroxy-1-(3-methoxyphenyl)ethyl)isoindolin-1-one as a yellow oil. (ES, m/z): 362.0, 364.0 [M+H, M+H+2]⁺.

### Step B: (S)-2-(2-(allyloxy)-1-(3-methoxyphenyl)ethyl)-6-bromoisoindolin-1-one

6-Bromo-2-(2-hydroxy-1-(3-methoxyphenyl)ethyl)isoindolin-1-one (1 g, 2.761 mmol, 1 equiv) was dissolved in acetonitrile (15 ml), then Ag₂O (3.2 g, 13.804 mmol, 5 equiv) and 3-iodoprop-1-ene (2.3 g, 13.804 mmol, 5.00 equiv) were added slowly, and the resulting mixture was stirred overnight at room temperature. Ag₂O was removed by filtration. The residue was applied onto a silica gel column with methanol/dichloromethane (0-5%) to yield (S)-2-(2-(allyloxy)-1-(3-methoxyphenyl)ethyl)-6-bromoisoindolin-1-one as a yellow oil. (ES, m/z): 402.1, 404.0 [M+H, M+H+2]⁺.

### Step C: 3-allyl-2-((S)-2-(allyloxy)-1-(3-methoxyphenyl)ethyl)-6-bromoisoindolin-1-one

To a mixture of (S)-2-(2-(allyloxy)-1-(3-methoxyphenyl)ethyl)-6-bromoisoindolin-1-one (2.1 g, 5.220 mmol, 1.00 equiv) in tetrahydrofuran (40 mL) was added sodium hydride (1.044 g, 26.101 mmol, 5.00 equiv) at 0° C., and the resulting mixture was stirred at 0° C. for 0.5 h. Allyl bromide (3.158 g, 26.101 mmol, 5.00 equiv) was added to the mixture and the mixture was stirred at 85° C. for 3 h. After cooling down to room temperature, the reaction was quenched with H₂O and extracted with EtOAc twice. The organic layers were combined, dried over Na₂SO₄, filtered and concentrated. The resulting residue was purified by silica gel chromatography (0-50% EtOAc/petroleum ether) to yield 3-allyl-2-((S)-2-(allyloxy)-1-(3-methoxyphenyl)ethyl)-6-bromoisoindolin-1-one as a yellow oil. (ES, m/z): 442.2, 444.2 [M+H, M+H+2].

### Step D: (1S,Z)-10-bromo-1-(3-methoxyphenyl)-1,2,7,7a-tetrahydro-[1,4]oxazonino[5,4-a]isoindol-12(4H)-one

A solution of 3-allyl-2-((S)-2-(allyloxy)-1-(3-methoxyphenyl)ethyl)-6-bromoisoindolin-1-one (1.253 g, 2.832 mmol, 1.00 equiv) and Grubbs catalyst 1st generation (142 mg, 0.170 mmol, 0.06 equiv) in dichloromethane (30 mL) was stirred at room temperature overnight under atmosphere of nitrogen. The resulting mixture was concentrated and the residue was purified by silica gel chromatography (0-50% EtOAc/petroleum ether) to yield (1S,Z)-10-bromo-1-(3-methoxyphenyl)-1,2,7,7a-tetrahydro-[1,4]oxazonino[5,4-a]isoindol-12(4H)-one as a yellow solid. (ES, m/z): 436.2, 438.2 [M+Na, M+Na+2]⁺.

### Step E: (1S,Z)-1-(3-methoxyphenyl)-10-(1H-pyrazol-4-yl)-1,2,7,7a-tetrahydro-[1,4]oxazonino[5,4-a]isoindol-12(4H)-one

The mixture of (1S,Z)-10-bromo-1-(3-methoxyphenyl)-1,2,7,7a-tetrahydro-[1,4]oxazonino[5,4-a]isoindol-12(4H)-one (350 mg, 0.845 mmol, 1.00 equiv), tert-butyl 4-(4,4,5,5-tetramethyl-1,3,2-dioxaborolan-2-yl)-1H-pyrazole-1-carboxylate (298 mg, 1.014 mmol, 1.20 equiv), potassium carbonate (350 mg, 2.534 mmol, 3.00 equiv) and tetrakis(triphenylphosphine)palladium(0) (97 mg, 0.084 mmol, 0.10 equiv) in 1,4-dioxane (5 mL) and water (1 mL) was stirred at 95° C. under atmosphere of nitrogen overnight. After cooling down to room temperature, the reaction was quenched with H₂O and extracted with EtOAc twice. The organic layers were combined, dried over Na₂SO₄, filtered and concentrated. The residue was purified by reverse phase chromatography on C₁₈ column (80 g, MeCN/H₂O (0.05% CF₃COOH): 0>>>45%) to yield (1S,Z)-1-(3-methoxyphenyl)-10-(1H-pyrazol-4-yl)-1,2,7,7a-tetrahydro-[1,4]oxazonino[5,4-a]isoindol-12(4H)-one as a white solid.

¹H-NMR (DMSO, 300 MHz): δ 8.15 (s, 2H), 7.77-7.89 (m, 2H), 7.53 (d, J=7.9 Hz, 1H), 7.28 (t, J=7.9 Hz, 1H), 6.94-7.06 (m, 2H), 6.83-6.92 (m, 1H), 5.41-5.46 (m, 1H), 5.37 (d, J=11.5 Hz, 1H), 4.62-4.75 (m, 1H), 4.42-4.60 (m, 2H), 4.30 (t, J=3.6 Hz, 1H), 3.89 (dd, J=16.7, 4.6 Hz, 1H), 3.70 (s, 3H), 3.62 (dd, J=12.7, 3.0 Hz, 1H), 3.48 (td, J=12.3, 4.6 Hz, 1H), 2.36 (d, J=13.3 Hz, 1H). ES, m/z): 402.2 [M+H]⁺.

### Example 2: Compound #11

### (1S)-1-(3-methoxyphenyl)-10-(1H-pyrazol-4-yl)-1,2,5,6,7,7a-hexahydro-[1,4]oxazonino[5,4-a]isoindol-12(4H)-one

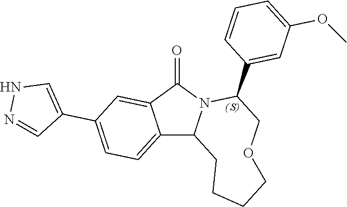

A mixture of (1S,Z)-1-(3-methoxyphenyl)-10-(1H-pyrazol-4-yl)-1,2,7,7a-tetrahydro-[1,4]oxazonino[5,4-a]isoindol-12(4H)-one (60 mg, 0.149 mmol, 1.00 equiv) and Pd/C (30 mg) in MeOH (2 mL) was stirred at room temperature for 1 h under atmosphere of hydrogen. After filtration, the filtrate was concentrated and the residue was purified by Prep-HPLC to yield (1S)-1-(3-methoxyphenyl)-10-(1H-pyrazol-4-yl)-1,2,5,6,7,7a-hexahydro-[1,4]oxazonino[5,4-a]isoindol-12(4H)-one as an off-white solid.

¹H-NMR (DMSO, 300 MHz): δ 8.16 (s, 2H), 7.92 (d, J=1.6 Hz, 1H), 7.82 (dd, J=7.9, 1.7 Hz, 1H), 7.40 (d, J=7.9 Hz, 1H), 7.27 (t, J=7.9 Hz, 1H), 6.97-7.05 (m, 2H), 6.87 (dd, J=8.1, 2.5 Hz, 1H), 5.37 (s, 1H), 4.42 (d, J=12.9 Hz, 1H), 4.18-4.24 (m, 1H), 3.75-3.83 (m, 1H), 3.64-3.74 (m, 4H), 3.45 (d, J=11.9 Hz, 1H), 2.48-2.60 (m, 1H), 1.80 (d, J=14.6 Hz, 1H), 1.27 (d, J=10.3 Hz, 2H), 0.86-1.04 (m, 2H). LC-MS (ES, m/z): 404.2 [M+H]⁺.

### Example 3: Compound #3

### (7S*)-7-(3-methoxyphenyl)-3-(1H-pyrazol-4-yl)-7,8,9,10,11,11a-hexahydro-5H-azepino[2,1-a]isoindol-5-one

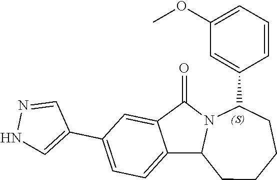

### And Example 4: Compound #1

### (7R*)-7-(3-methoxyphenyl)-3-(1H-pyrazol-4-yl)-7,8,9,10,11,11a-hexahydro-5H-azepino[2,1-a]isoindol-5-one

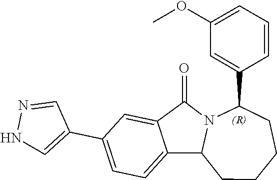

### Step A: (E)-N-(3-methoxybenzylidene)-2-methylpropane-2-sulfinamide

To a solution of 3-methoxybenzaldehyde (6 g, 44.1 mmol, 1.0 equiv) in THF (100 mL) was added titanium (IV) ethoxide (20.1 g, 88.1 mmol, 2.0 equiv), 2-methylpropane-2-sulfinamide (5.87 g, 48.4 mmol, 1.1 equiv) under N₂ at room temperature. The reaction was stirred overnight at 60° C., then quenched with NaHCO₃ (aq.). The solid was filtered out and the resulting mixture was extracted with ethyl acetate, dried over Na₂SO₄ and concentrated under vacuum. The residue was purified by column on silica gel with ethyl acetate/petroleum ether (0-30%) to yield N-(3-methoxybenzylidene)-2-methylpropane-2-sulfinamide as a yellow oil. LC/MS: mass Calculated. for C₁₂H₁₇NO₂S: 239.33, Measured: 479.0 [2M+H]⁺.

### Step B: N-(1-(3-methoxyphenyl)but-3-en-1-yl)-2-methylpropane-2-sulfinamide

To a solution of N-(3-methoxybenzylidene)-2-methylpropane-2-sulfinamide (6 g, 25.1 mmol, 1.0 equiv) in THF (50 mL) was added allylmagnesium bromide (1M in THF) (50 mL, 50 mmol, 2.0 equiv) dropwise under N₂ at 0° C. The reaction was stirred overnight at room temperature, then quenched with water, extracted with ethyl acetate, dried over Na₂SO₄ and concentrated under vacuum. The residue was purified by column on silica gel with ethyl acetate/petroleum ether (0-80%) to yield N-(1-(3-methoxyphenyl)but-3-enyl)-2-methylpropane-2-sulfinamide as a light yellow oil. TLC: R_{f}=0.3 (ethyl acetate/petroleum ether=1/1)

### Step C: 1-(3-methoxyphenyl)but-3-en-1-amine

To a solution of N-(1-(3-methoxyphenyl)but-3-enyl)-2-methylpropane-2-sulfinamide (1.1 g, 3.9 mmol, 1.0 equiv) in MeOH (5 mL) was added hydrogen chloride solution in 1,4-dioxane (5 mL, 20 mmol, 5 equiv). The reaction was stirred for 1 h at room temperature, then quenched with NaHCO₃ (aq.) (30 mL), extracted with ethyl acetate, dried over Na₂SO₄ and concentrated under vacuum. The residue was purified by column on silica gel with MeOH/DCM (0-10%) to yield 1-(3-methoxyphenyl)but-3-en-1-amine as a light yellow oil. LC/MS: mass Calculated. for C₁₁H₁₅NO: 177.24, Measured: 178.2 [M+H]⁺.

### Step D: 6-bromo-2-(1-(3-methoxyphenyl)but-3-en-1-yl)isoindolin-1-one

To a solution of 1-(3-methoxyphenyl)but-3-en-1-amine (650 mg, 3.667 mmol, 1.0 equiv) in acetonitrile (20 mL) was added methyl 5-bromo-2-(bromomethyl)benzoate (1.13 g, 3.669 mmol, 1.001 equiv), N,N-Diisopropylethylamine (1.42 g, 10.987 mmol, 2.996 equiv) under N₂. The reaction was stirred overnight at 90° C., then quenched with water, extracted with ethyl acetate, dried over Na₂SO₄ and concentrated under vacuum. The residue was purified by column on silica gel with ethyl acetate/petroleum ether (0-50%) to yield 6-bromo-2-(1-(3-methoxyphenyl)but-3-enyl)isoindolin-1-one as a light yellow oil. LC/MS: 371.9 [M+H]⁺ found.

### Step E: 3-allyl-6-bromo-2-(1-(3-methoxyphenyl)but-3-en-1-yl)isoindolin-1-one

To a solution of 6-bromo-2-[1-(3-methoxyphenyl)but-3-en-1-yl]-2,3-dihydro-1H-isoindol-1-one (500 mg, 1.34 mmol, 1.0 equiv), NaH (269 mg, 6.7 mmol, 5.0 equiv, 60%) in tetrahydrofuran (10 ml), was added allyl bromide (812 mg, 6.72 mmol, 5.0 equiv). The resulting solution was stirred for 50 min at 75° C. The reaction was then quenched by the addition of water. The resulting solution was extracted with ethyl acetate and the organic layers combined. The resulting mixture was washed with water and brine. The residue was purified by silica gel chromatography (0-50% PE/EA) to yield 3-allyl-6-bromo-2-(1-(3-methoxyphenyl)but-3-en-1-yl)isoindolin-1-one as a yellow solid. LC/MS: mass Calculated. for C₂₂H₂₂BrNO₂: 412.32, Measured: 412.0 [M+H]⁺.

### Step F: 3-bromo-7-(3-methoxyphenyl)-7,8,11,11a-tetrahydro-5H-azepino[2,1-a]isoindol-5-one

To a solution of 6-bromo-2-[1-(3-methoxyphenyl)but-3-en-1-yl]-3-(prop-2-en-1-yl)-2,3-dihydro-1H-isoindol-1-one (120 mg, 0.29 mmol, 1.0 equiv) in dichloromethane (5 ml), Grubbs catalyst 1st generation (24 mg, 0.03 mmol, 0.1 equiv) was added. The resulting solution was stirred over night at room temperature. The mixture was filtered through a CELITE® pad. The filtrate was concentrated under vacuum. The residue was applied on a silica gel column and eluted with PE/EA (1/50%) to yield 3-bromo-7-(3-methoxyphenyl)-7,8,11,11a-tetrahydro-5H-azepino[2,1-a]isoindol-5-one as a brown solid. LC/MS: mass Calculated. for C₂₀H₁₈BrNO₂: 384.266, Measured: 384.1 [M+H]⁺.

### Step G: 7-(3-methoxyphenyl)-3-(1H-pyrazol-4-yl)-7,8,11,11a-tetrahydro-5H-azepino[2,1-a]isoindol-5-one

To a solution of 3-bromo-7-(3-methoxyphenyl)-7,8,11,11a-tetrahydro-5H-azepino[2,1-a]isoindol-5-one (160 mg, 0.42 mmol, 1.0 equiv), tert-butyl 4-(tetramethyl-1,3,2-dioxaborolan-2-yl)-1H-pyrazole-1-carboxylate (147 mg, 0.5 mmol, 1.2 equiv), potassium carbonate (172 mg, 1.25 mmol, 3.0 equiv), and tetrakis(triphenylphosphine)palladium(0) (97 mg, 0.084 mmol) in 1,4-dioxane/H₂O (5.5 ml, 10/1). The resulting solution was stirred over night at 85° C. The reaction was then quenched by the addition of brine. The resulting solution was extracted with DCM and the organic layers combined. The resulting mixture was washed with water and brine. The resulting residue was purified by silica gel chromatography (0-10% DCM/MeOH) to yield 7-(3-methoxyphenyl)-3-(1H-pyrazol-4-yl)-7,8,11,11a-tetrahydro-5H-azepino[2,1-a]isoindol-5-one as a white solid. LC/MS: mass Calculated. for C₂₃H₂₁N₃O₂: 371.432, Measured: 372.2 [M+H]⁺.

### Step E: (7S*)-7-(3-methoxyphenyl)-3-(1H-pyrazol-4-yl)-7,8,9,10,11,11a-hexahydro-5H-azepino[2,1-a]isoindol-5-one; and (7R*)-7-(3-methoxyphenyl)-3-(1H-pyrazol-4-yl)-7,8,9,10,11,11a-hexahydro-5H-azepino[2,1-a]isoindol-5-one

To a solution of 7-(3-methoxyphenyl)-3-(1H-pyrazol-4-yl)-7,8,11,11a-tetrahydro-5H-azepino[2,1-a]isoindol-5-one (60 mg, 0.162 mmol, 1.0 equiv) in tetrahydrofuran (5 ml), palladium on activated carbon (60 mg, 5%) was added. The mixture was stirred 1 h at room temperature under an atmosphere of hydrogen. The solid was filtered out. The filtrate was concentrated under vacuum. The resulting residue was purified by chromatography on a reverse phase C₁₈ column (0-70% water/CH₃CN), followed by chiral HPLC purification to yield (7S*)-7-(3-methoxyphenyl)-3-(1H-pyrazol-4-yl)-7,8,9,10,11,11a-hexahydro-5H-azepino[2,1-a]isoindol-5-one. LC/MS: mass Calculated. for C₂₃H₂₁N₃O₂: 371.432, Measured: 372.2 [M+H]⁺.

and (7R*)-7-(3-methoxyphenyl)-3-(1H-pyrazol-4-yl)-7,8,9,10,11,11a-hexahydro-5H-azepino[2,1-a]isoindol-5-one. LC/MS: mass Calculated. for C₂₃H₂₃N₃O₂: 373.448, Measured: 374.2 [M+H]⁺.

¹H NMR (300 MHz, Methanol-d4) δ 7.84-8.26 (m, 4H), 7.54-7.74 (m, 2H), 7.26 (t, J=7.9 Hz, 1H), 6.79 (m, J=10.2, 5.8, 2.1 Hz, 3H), 5.36 (t, J=3.8 Hz, 1H), 4.99 (d, J=8.7 Hz, 1H), 3.78 (s, 3H), 2.55-2.66 (m, 1H), 2.26-2.42 (m, 2H), 1.96 (m, J=8.7, 4.4 Hz, 1H), 1.62-1.84 (m, 3H), 1.28-1.39 (m, 3H).

### Example 5: Compound #15

### (Rac)-1-(3-methoxyphenyl)-8-(1H-pyrazol-4-yl)-1,2,3,4,5,5a-hexahydro-10H-[1,2]diazepino[7,1-a]isoindol-10-one

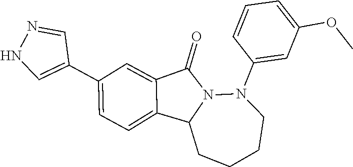 [SMILES: COc1cccc(N2CCCCC3c4ccc(-c5cn[nH]c5)cc4C(=O)N32)c1]

To a solution of 1-(3-methoxyphenyl)-8-(1H-pyrazol-4-yl)-1,2,5,5a-tetrahydro-10H-[1,2]diazepino[7,1-a]isoindol-10-one (80 mg, 0.215 mmol, 1.0 equiv) in tetrahydrofuran (5 ml), palladium on activated carbon (40 mg, 5%) was added. The mixture was stirred 1 h at room temperature under an atmosphere of hydrogen. The solid was filtered out. The filtrate was concentrated under vacuum. The resulting residue was purified by chromatography on a reverse phase C₁₈ column (0-70% water/CH₃CN) to yield (Rac)-1-(3-methoxyphenyl)-8-(1H-pyrazol-4-yl)-1,2,3,4,5,5a-hexahydro-10H-[1,2]diazepino[7,1-a]isoindol-10-one as a white solid.

¹H NMR (400 MHz, DMSO-d6) δ 13.01 (s, 1H), 8.35 (s, 1H), 8.04 (s, 1H), 7.90-7.98 (m, 2H), 7.59 (d, J=7.8 Hz, 1H), 7.10 (t, J=8.2 Hz, 1H), 6.33 (ddd, J=8.5, 6.6, 2.3 Hz, 2H), 6.24 (t, J=2.3 Hz, 1H), 4.98 (t, J=5.4 Hz, 1H), 3.96 (dt, J=14.3, 4.7 Hz, 1H), 3.67 (s, 3H), 3.27-3.41 (m, 1H), 2.04 (ddt, J=26.6, 20.0, 10.1 Hz, 2H), 1.80-1.91 (m, 1H), 1.49-1.68 (m, 2H), 1.14 (q, J=10.9 Hz, 1H).

LC/MS: mass Calculated. for C₂₂H₂₂N₄O₂: 374.436, Measured: 375.1 [M+H]⁺.

### Example 6: Compound #16

### 1-(3-methoxyphenyl)-8-(1H-pyrazol-4-yl)-1,2,5,5a-tetrahydro-10H-[1,2]diazepino[7,1-a]isoindol-10-one

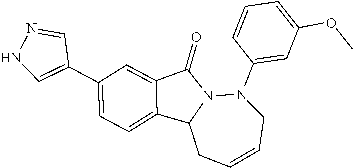 [SMILES: COc1cccc(N2CC=CCC3c4ccc(-c5cn[nH]c5)cc4C(=O)N32)c1]

### Step A: 6-bromo-2-((3-methoxyphenyl)amino)isoindolin-1-one

To a solution of 2-amino-6-bromo-2,3-dihydro-1H-isoindol-1-one (2 g, 8.8 mmol, 1.0 equiv), 3-methoxy phenyl boric acid (1.6 g, 10.6 mmol, 1.2 equiv) in dichloromethane (30 ml), triethylamine (3.7 ml), anhydrous Cu(OAc)₂ (1.6 g, 8.8 mmol, 1.0 equiv) was added. The resulting solution was stirred overnight at room temperature. The mixture was filtered through a CELITE® pad. The filtrate was concentrated under vacuum. The residue was applied on a silica gel column and eluted with DCM/MeOH. (1/20) to yield 6-bromo-2-((3-methoxyphenyl)amino)isoindolin-1-one as a yellow solid. LC/MS: mass Calculated. for C₁₅H₁₃BrN₂O₂: 333.18, Measured: 333.1 [M+H]⁺.

### Step B: 3-allyl-2-(allyl(3-methoxyphenyl)amino)-6-bromoisoindolin-1-one

To a solution of 6-bromo-2-[(3-methoxyphenyl)amino]-2,3-dihydro-1H-isoindol-1-one (400 mg, 1.2 mmol, 1.0 equiv), sodium hydride (240 mg, 6 mmol, 5.0 equiv, 60%) in tetrahydrofuran (10 ml), allyl bromide (726 mg, 6 mmol, 5.0 equiv) was added. The resulting solution was stirred for 30 min at room temperature and then stirred for 1 h at 70° C. The reaction was then quenched by the addition of water. The resulting solution was extracted with ethyl acetate and the organic layers combined. The resulting mixture was washed with water and brine. The residue was purified by silica gel chromatography (0-50% PE/EA) to yield 3-allyl-2-(allyl(3-methoxyphenyl)amino)-6-bromoisoindolin-1-one as a colorless oil. LC/MS: mass Calculated. for C₂₁H₂₁BrN₂O₂: 413.308, Measured: 413.1 [M+H]⁺.

### Step C: 8-bromo-1-(3-methoxyphenyl)-1,2,5,5a-tetrahydro-10H-[1,2]diazepino[7,1-a]isoindol-10-one

To a solution of 6-bromo-2-[(3-methoxyphenyl)(prop-2-en-1-yl)amino]-3-(prop-2-en-1-yl)-2,3-dihydro-1H-isoindol-1-one (170 mg, 0.4 mmol, 1.0 equiv) in dichloromethane (5 ml), Grubbs catalyst 1st generation (33.9 mg, 0.04 mmol, 0.1 equiv) was added. The resulting solution was stirred over night at room temperature. The mixture was filtered through a CELITE® pad. The filtrate was concentrated under vacuum. The residue was applied on a silica gel column and eluted with PE/EA (1/50%) to yield 8-bromo-1-(3-methoxyphenyl)-1,2,5,5a-tetrahydro-10H-[1,2]diazepino[7,1-a]isoindol-10-one as a black oil. LC/MS: mass Calculated. for C₁₉H₁₇BrN₂O₂: 385.254, Measured: 384.9 [M+H]⁺.

### Step D: 1-(3-methoxyphenyl)-8-(1H-pyrazol-4-yl)-1,2,5,5a-tetrahydro-10H-[1,2]diazepino[7,1-a]isoindol-10-one

To a solution of 8-bromo-1-(3-methoxyphenyl)-1H,2H,5H,5aH,10H-[1,2]diazepino[3,2-a]isoindol-10-one (270 mg, 0.7 mmol, 1.0 equiv), tert-butyl 4-(tetramethyl-1,3,2-dioxaborolan-2-yl)-1H-pyrazole-1-carboxylate (247 mg, 0.8 mmol, 1.2 equiv), potassium carbonate (290 mg, 2 mmol, 3.0 equiv) and Pd(PPh₃)₄ (46 mg, 0.040 mmol) in 1,4-dioxane/H₂O (11 ml, 10/1). The resulting solution was stirred over night at 85° C. The reaction was then quenched by the addition of brine. The resulting solution was extracted with DCM and the organic layers combined. The resulting mixture was washed with water and brine. The residue was purified by silica gel chromatography (0-10% DCM/MeOH) to yield 1-(3-methoxyphenyl)-8-(1H-pyrazol-4-yl)-1,2,5,5a-tetrahydro-10H-[1,2]diazepino[7,1-a]isoindol-10-one as a white solid.

LC/MS: mass Calculated. for C₂₂H₂₀N₄O₂: 372.42, Measured: 373.0 [M+H]⁺.

¹H NMR (400 MHz, DMSO-d6) δ 13.03 (s, 1H), 8.37 (s, 1H), 8.06 (s, 2H), 7.95 (d, J=6.5 Hz, 2H), 7.65 (d, J=8.5 Hz, 1H), 7.14 (t, J=8.2 Hz, 1H), 6.34-6.47 (m, 3H), 5.82 (dt, J=9.1, 4.1 Hz, 1H), 5.49-5.60 (m, 1H), 5.02 (t, J=5.1 Hz, 1H), 4.56 (d, J=16.4 Hz, 1H), 4.19 (dd, J=16.4, 4.9 Hz, 1H), 3.70 (s, 3H), 3.01 (dt, J=13.2, 6.2 Hz, 1H), 2.55 (s, 2H), 1.77 (d, J=15.6 Hz, 1H), 1.22 (dt, J=22.5, 13.0 Hz, 2H).

### Example 7: Compound #6

### (7R*)-7-(3-hydroxyphenyl)-3-(1H-pyrazol-4-yl)-7,8,9,10,11,11a-hexahydro-5H-azepino[2,1-a]isoindol-5-one

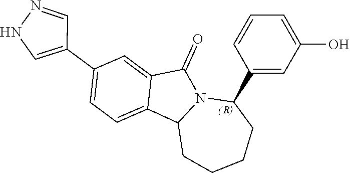

### Step A: (7R*)-7-(3-methoxyphenyl)-3-(1H-pyrazol-4-yl)-7,8,11,11a-tetrahydro-5H-azepino[2,1-a]isoindol-5-one

To a solution of (5R*)-9-bromo-5-(3-methoxyphenyl)-1H,4H,5H,7H,11bH-azepino[2,1-a]isoindol-7-one (153 mg, 0.398 mmol, 1.0 equiv) in DMF/H₂O (7.5 mL, 4:1) at room temperature was added K₂CO₃ (164.839 mg, 1.194 mmol, 3.0 equiv), tert-butyl 4-(tetramethyl-1,3,2-dioxaborolan-2-yl)-1H-pyrazole-1-carboxylate (140.545 mg, 0.478 mmol, 1.2 equiv), Pd(PPh₃)₄ (45.988 mg, 0.040 mmol, 0.1 equiv) under N₂. The reaction mixture was then stirred at 80° C. overnight. The reaction was quenched with water and extracted with EtOAc three times. The combined organic layer was washed with water, dried over Na₂SO₄, filtered and concentrated. The residue was applied onto a silica gel column with ethyl acetate/petroleum ether to (7R*)-7-(3-methoxyphenyl)-3-(1H-pyrazol-4-yl)-7,8,11,11a-tetrahydro-5H-azepino[2,1-a]isoindol-5-one as a yellow oil. LC-MS: ES, m/z): 372.3[M+H]⁺.

### Step B: (7R*)-7-(3-methoxyphenyl)-3-(1H-pyrazol-4-yl)-7,8,9,10,11,11a-hexahydro-5H-azepino[2,1-a]isoindol-5-one

To (7R*)-7-(3-methoxyphenyl)-3-(1H-pyrazol-4-yl)-7,8,11,11a-tetrahydro-5H-azepino[2,1-a]isoindol-5-one (120 mg, 0.323 mmol, 1 equiv) in anhydrous MeOH (6 ml) at room temperature was added Pd/C (68.492 mg, 0.646 mmol, 2.0 equiv). To the resulting mixture, hydrogen was introduced. The resulting solution was stirred for 3 h at room temperature. The solids were filtered out. The resulting mixture was concentrated under vacuum. The resulting residue comprising (7S)-7-(3-methoxyphenyl)-3-(1H-pyrazol-4-yl)-7,8,9,10,11,11a-hexahydro-5H-azepino[2,1-a]isoindol-5-one was used in the next step without further purification. LC-MS: (ES, m/z): 374.3 [M+H]⁺.

### Step C: (7R*)-7-(3-hydroxyphenyl)-3-(1H-pyrazol-4-yl)-7,8,9,10,11,11a-hexahydro-5H-azepino[2,1-a]isoindol-5-one

To a solution of (7R*)-7-(3-methoxyphenyl)-3-(1H-pyrazol-4-yl)-7,8,9,10,11,11a-hexahydro-5H-azepino[2,1-a]isoindol-5-one (120 mg, 0.321 mmol, 1 equiv) in DCM (5 mL) at −78° C. was added BBr₃ in DCM (1.607 mL, 1.607 mmol, 1 M, 5 equiv) and the resulting mixture was stirred for 2 h, from −78° C. to room temperature. The reaction was quenched with ice water and extracted with DCM three time. The combined organic layer was dried over Na₂SO₄, filtered and concentrated. The residue was applied onto a reverse phase C₁₈ column with H₂O/CH₃CN (68%) to yield (7R*)-7-(3-hydroxyphenyl)-3-(1H-pyrazol-4-yl)-7,8,9,10,11,11a-hexahydro-5H-azepino[2,1-a]isoindol-5-one as a white solid.

LC-MS: (ES, m/z): 360.2[M+H]⁺.

¹H-NMR: (300 MHz, DMSO-d6): δ9.13 (s, 1H), 8.13 (d, J=8.2 Hz, 2H), 7.85 (d, J=8.1 Hz, 1H), 7.76 (d, J=10.6 Hz, 1H), 7.61 (dd, J=10.4, 7.8 Hz, 1H), 7.11-6.93 (m, 1H), 6.62-6.40 (m, 3H), 5.14 (d, J=4.0 Hz, 1H), 4.86 (d, J=8.7 Hz, 1H), 2.40 (d, J=16.1 Hz, 1H), 2.17 (q, J=6.8 Hz, 2H), 1.80 (d, J=13.2 Hz, 1H), 1.52 (p, J=14.7, 13.0 Hz, 3H), 1.12 (q, J=10.8 Hz, 1H).

### Example 8: Compound #13

### (9R*)-9-(3-methoxyphenyl)-2-(1H-pyrazol-4-yl)-4b,5,6,7,8,9-hexahydro-11H-pyrido[3′,2′:3,4]pyrrolo[1,2-a]azepin-11-one

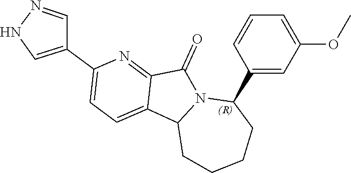

### Step A: 2-chloro-6-(1-(3-methoxyphenyl)but-3-enyl)-5,6-dihydropyrrolo[3,4-b]pyridin-7-one

A solution of methyl 3-(bromomethyl)-6-chloropyridine-2-carboxylate (1.0 g, 3.781 mmol, 1.00 eq), 1-(3-methoxyphenyl)but-3-en-1-amine (0.970 g, 4.537 mmol, 1.2 eq) and DIEA (2.443 g, 18.903 mmol, 5.0 eq) in CH₃CN (20 mL) was stirred at room temperature for 30 min with an inert atmosphere of nitrogen. The reaction mixture was then stirred overnight, under refluxing. The reaction mixture was poured into water, extracted with EtOAc, washed with brine and concentrated. The residue was applied onto a silica gel EA/PE (40%) to yield 2-chloro-6-(1-(3-methoxyphenyl)but-3-enyl)-5,6-dihydropyrrolo[3,4-b]pyridin-7-one as yellow solid.

### Step B: 5-allyl-2-chloro-6-(1-(3-methoxyphenyl)but-3-enyl)-5,6-dihydropyrrolo[3,4-b]pyridin-7-one

To a mixture of 2-chloro-6-(1-(3-methoxyphenyl)but-3-enyl)-5,6-dihydropyrrolo[3,4-b]pyridin-7-one (770 mg, 2.342 mmol, 1.00 equiv) in tetrahydrofuran (10 mL) was added sodium hydride (112 mg, 2.810 mmol, 1.20 equiv, 60%) at 0° C., and the mixture was stirred at 0° C. for 1 h. Allyl bromide (311 mg, 2.576 mmol, 1.10 equiv) was added and the resulting mixture was stirred at 90° C. for 2 h. After cooling the mixture to room temperature, the reaction was quenched with H₂O and extracted with EtOAc twice. The organic layers were combined, dried over Na₂SO₄, filtered and concentrated. The resulting residue was purified by silica gel chromatography (0-50% EtOAc/petroleum ether) to yield 5-allyl-2-chloro-6-(1-(3-methoxyphenyl)but-3-enyl)-5,6-dihydropyrrolo[3,4-b]pyridin-7-one as a white solid. LC/MS: mass Calculated for C₂₁H₂₁ClN₂O₂: 368.1, Measured: 369.2 [M+H]+.

### Step C: (10R*)-5-chloro-10-(3-methoxyphenyl)-6,9-diazatricyclo[7.5.0.02,7]tetradeca-2,4,6,12-tetraen-8-one and (10S*)-5-chloro-10-(3-methoxyphenyl)-6,9-diazatricyclo[7.5.0.02,7]tetradeca-2,4,6,12-tetraen-8-one

A solution of 5-allyl-2-chloro-6-(1-(3-methoxyphenyl)but-3-enyl)-5,6-dihydropyrrolo[3,4-b]pyridin-7-one (480 mg, 1.301 mmol, 1.00 equiv) and grubbs catalyst 1st generation (109 mg, 0.130 mmol, 0.10 equiv) in dichloromethane (10 mL) was stirred overnight at room temperature and under nitrogen. The resulting mixture was concentrated and the residue was purified by silica gel chromatography (0-50% EtOAc/petroleum ether) to yield (10R*)-5-chloro-10-(3-methoxyphenyl)-6,9-diazatricyclo[7.5.0.02,7]tetradeca-2,4,6,12-tetraen-8-one as a light yellow solid and (10S*)-5-chloro-10-(3-methoxyphenyl)-6,9-diazatricyclo[7.5.0.02,7]tetradeca-2,4,6,12-tetraen-8-one as a light yellow solid. LC/MS: mass Calculated. for C₁₉H₁₉ClN₂O₂: 340.1, Measured: 341.2 [M+H]+.

### Step D: (10R*)-10-(3-methoxyphenyl)-5-(1H-pyrazol-4-yl)-6,9-diazatricyclo[7.5.0.02,7]tetradeca-2,4,6,12-tetraen-8-one

A mixture of (10R*)-5-chloro-10-(3-methoxyphenyl)-6,9-diazatricyclo[7.5.0.02,7]tetradeca-2,4,6,12-tetraen-8-one (190 mg, 0.558 mmol, 1.00 equiv), tert-butyl 4-(4,4,5,5-tetramethyl-1,3,2-dioxaborolan-2-yl)-1H-pyrazole-1-carboxylate (197 mg, 0.669 mmol, 1.20 equiv), potassium carbonate (231 mg, 1.673 mmol, 3.00 equiv) and tetrakis(triphenylphosphine)palladium(0) (64 mg, 0.056 mmol, 0.10 equiv) in 1,4-dioxane (3 mL) and water (1 mL) was stirred overnight at 95° C. and under atmosphere of nitrogen. The resulting mixture was then cooled to room temperature, and the reaction was quenched with H₂O and extracted with EtOAc twice. The organic layers were combined, dried over Na₂SO₄, filtered and concentrated. The resulting residue was purified by silica gel chromatography (0-50% EtOAc/petroleum ether) to yield (10R*)-10-(3-methoxyphenyl)-5-(1H-pyrazol-4-yl)-6,9-diazatricyclo[7.5.0.02,7]tetradeca-2,4,6,12-tetraen-8-one as a light yellow solid. LC/MS: mass Calculated. for C₂₂H₂₀N₄O₂: 372.2, Measured: 373.2 [M+H]+.

### Step E: (10R)-10-(3-methoxyphenyl)-5-(1H-pyrazol-4-yl)-6,9-diazatricyclo[7.5.0.02,7]tetradeca-2,4,6-trien-8-one

A mixture of (10R*)-10-(3-methoxyphenyl)-5-(1H-pyrazol-4-yl)-6,9-diazatricyclo[7.5.0.02,7]tetradeca-2,4,6,12-tetraen-8-one (100 mg, 0.269 mmol, 1.00 equiv) and Pd/C (30 mg) in methanol (2 mL) was stirred at room temperature for 1 h under hydrogen. After filtration, the filtrate was concentrated and the resulting residue was purified by reverse phase chromatography on Cis column (80 g, MeCN/H₂O (0.05% CF3COOH): 0>>>45%) to yield (10R)-10-(3-methoxyphenyl)-5-(1H-pyrazol-4-yl)-6,9-diazatricyclo[7.5.0.02,7]tetradeca-2,4,6-trien-8-one as an off-white solid

LC/MS: mass Calculated. for C₂₂H₂₂N₄O₂: 374.2, Measured: 375.3 [M+H]+.

¹H NMR (400 MHz, DMSO-d6) δ 13.14 (s, 1H), 8.39 (s, 1H), 8.11 (d, J=8.0 Hz, 2H), 7.92 (d, J=8.2 Hz, 1H), 7.17 (t, J=8.1 Hz, 1H), 6.68-6.80 (m, 3H), 5.20 (d, J=10.4 Hz, 1H), 5.12 (dd, J=12.3, 4.7 Hz, 1H), 3.70 (s, 3H), 2.47 (s, 1H), 2.24 (dt, J=13.9, 6.0 Hz, 1H), 1.94 (dq, J=26.0, 13.5 Hz, 3H), 1.67 (q, J=12.5 Hz, 1H), 1.48 (q, J=12.0 Hz, 1H), 1.14 (q, J=12.3, 11.4 Hz, 1H).

### Biological Example 1

### hGRK2 LANCER In Vitro Assay

G-protein coupled receptor kinases (GR Kinases) desensitize activated G-protein coupled receptors (GPCRs), by phosphorylation of cytoplasmic loops or carboxyl-terminal tails of GPCRs. GRK2 is one of the 6 different GR kinases and is implicated in heart failure and diabetes.

The purpose of the LANCE Ultra assay (http://www.perkinelmer.com/Resources/TechnicalResources/ApplicationSupportKnowledgebase/LANCE/lance.xhtml) is used to test inhibitors against GRK2 in its inactive state. This assay is sensitive and requires as low as 10 nM enzyme, in a total volume of 10 μL. In addition, the ATP concentration can be varied over a broad range, without interfering with the assay or changing the assay condition. This property makes it easy to characterize very potent ATP-competitive inhibitors by increasing ATP concentrations. Testing inhibitors routinely at both high and low ATP concentrations also enables identification of potential non-ATP competitive inhibitors.

Paroxetine was used as the reference compound in this assay. The IC₅₀ value determined by the LANCE Ultra assay was 8.3 μM, which is in good agreement with the literature value (THAL, D. M., et al. “Paroxetine is a direct inhibitor of G protein-coupled receptor kinase 2 and increases myocardial contractility”, ACS Chemical Biology, 2012, pp 1830, Vol. 7).

This assay measures IC₅₀ values of test compounds (inhibitors) by monitoring GRK2 enzymatic activity at varying inhibitor concentrations.

Test compounds were dissolved in DMSO at 1 mM and were 3-fold serial diluted. The compound DMSO solutions were then added (100 nL) into a plate well using an acoustic dispenser. To each well was then added 20 nM GRK2 (5 μL) in assay buffer (20 mM HEPES, pH 7.5, 10 mM MgCl₂, 0.001% Tween-20). The plate was sealed and centrifuged at 1000 rpm for 1 min. The plate and wells containing a mixture of GRK2 and test compound were incubated at ambient temperature for 30 min (prior to initializing the enzymatic reaction).

Enzyme reactions were initiated by the addition of 4.9 μL Substrates/Eu-Ab mix to each well. For assays at low ATP concentration (1×Kₘ value), the Substrate/Eu-Ab mix contains 60 μM ATP, 400 nM ULight-peptide (LANCE® Ultra ULight™-DNA Topoisomerase 2-alpha (Thr1342) Peptide), and 8 nM Eu-Ab (LANCE® Ultra Europium-anti-phospho-DNA Topoisomerase 2-alpha (Thr1342)) in the assay buffer. For assays at high ATP concentration (20×Kₘ value), the Substrate/Eu-Ab mix contains 1.2 mM ATP, 400 nM ULight-peptide, and 8 nM Eu-Ab in the assay buffer. Final concentrations of reagents in the assays were as follows: 20 mM HEPES, pH 7.5; 10 mM MgCl₂; 0.001% Tween-20 (w/v); 30 or 600 μM ATP; 200 nM ULight-peptide; 4 nM Eu-Ab; 10 nM GRK2; and 1% DMSO.

The plates were sealed and centrifuged at 1000 rpm for 1 min. For reactions at low ATP concentration (30 μM), reaction mixtures were incubated at ambient temperature for 120 min. For reactions at high ATP concentration (600 μM), reaction mixtures were incubated at ambient temperature for 60 min.

The enzyme reactions were quenched by addition of 10 μL of 12 mM EDTA in 1× LANCE detection solution to each well. The plates were then incubated at ambient temperature for 30 min. Time-resolved fluorescence signal of reactions were read on an EnVision or PHERAstar plate reader with the following parameters: Excitation wavelength=337 nm, emission wavelength (donor)=620 nm; emission wavelength (acceptor)=665 nm.

To calculate IC₅₀ values, compounds were serially diluted 3-fold and tested in 11-point dose responses. The raw HTRF data were converted to % active as follows:

% active=(sample−NC)/(PC−NC)*100

where NC is the mean of negative control (reactions without GRK2), and PC is the mean of positive control (reactions with GRK2 but without inhibitor). IC₅₀ values were determined from a 4-parameter fit, using the following equation:

Y=Bottom+(Top−Bottom)/(1+10^{((Log IC50-X)*Hill slope)}),

where X=log₁₀ of the compound concentration.

Representative compounds of the present invention were tested according to the procedure described in Biological Example 1, above, with results as listed in Table 2 below. Results are reported as the IC₅₀ value. Variability for the functional assay was typically within 2-fold.

TABLE 2
Biological Activity, Compounds of Formula (I)

| | GRK2 Lancer |
|---|---|
| ID No. | IC₅₀ (μM) |
| 1 | <0.005 |
| 2 | 0.008 |
| 3 | 0.009 |
| 4 | 0.009 |
| 5 | 0.011 |
| 6 | 0.012 |
| 7 | 0.024 |
| 8 | 0.166 |
| 9 | 1.404 |
| 12 | not tested |
| 13 | not tested |
| 14 | 1.014 |
| 15 | 0.006 |
| 16 | 0.008 |

### Biological Example 2—Prophetic Example

### GLP-1 Mediated Beta-Arrestin Recruitment Assay

Path Hunter® eXpress GLP1R CHO-K1 β-Arrestin cells are plated at 6000/well in a 384-well PDL white and opaque plate in F12 medium with 10% FBS, 0.3 mg/ml hygromycin, and 0.8 mg/ml G418. The plate is maintained in a humidified incubator at 37° C. and 5% CO₂ for 2 days before the experiment. On the day of the experiment, the cells are washed once with the Assay Buffer (HBSS with calcium and magnesium, 20 mM HEPES, and 0.1% fatty-acid free BSA). Test compound or vehicle (DMSO) is added to the cells at the indicated concentrations, 10 min prior to the addition of GLP-1. The final DMSO concentration is 0.1%. After 90 min incubation at 37° C., the detection reagent is added the cells, followed by 60 min incubation at the room temperature. The plate is read on MicroBeta LumiJet (PerkinElmer, Waltham, Mass.).

### Formulation Example 1

### Solid, Oral Dosage Form—Prophetic Example

As a specific embodiment of an oral composition, 100 mg of the Compound #1 (prepared for example, as in Example 4) is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

Throughout this application, various publications are cited. The disclosure of these publications is hereby incorporated by reference into this application to describe more fully the state of the art to which this invention pertains.

1. A compound of formula (I) 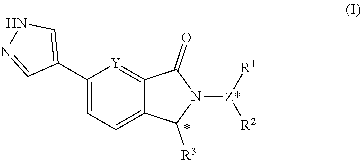
wherein
R¹ is phenyl; wherein the phenyl is optionally substituted with one to two substituents independently selected from the group consisting of halogen, hydroxy and C₁₋₂alkoxy;
Z is selected from the group consisting of CH and N;
R² and R³ are taken together with the atoms to which they are bound to form a fused ring structure selected from the group consisting of 1λ²-azepanyl, 2,3,6,7-tetrahydro-1λ²-azepinyl, 1,4λ²-oxazonanyl, (Z)-2,3,6,9-tetrahydro-5H-1,4λ²-oxazoninyl, 1λ²,2λ²-diazepanyl and 4,7-dihydro-3H-1λ²,2λ²-diazepinyl;
Y is selected from the group consisting of CH and N;
or a pharmaceutically acceptable salt thereof.

2. The compound of as in claim 1, wherein
R¹ is phenyl; wherein the phenyl is optionally substituted with one to two substituents independently selected from the group consisting of fluoro, hydroxy and methoxy;
Z is selected from the group consisting of CH and N;
R² and R³ are taken together with the atoms to which they are bound to form a fused ring structure selected from the group consisting of 1λ²-azepanyl, 2,3,6,7-tetrahydro-1λ²-azepinyl, 1,4λ²-oxazonanyl, (Z)-2,3,6,9-tetrahydro-5H-1,4λ²-oxazoninyl, 1λ²,2λ²-diazepanyl and 4,7-dihydro-3H-1λ²,2λ²-diazepinyl;
Y is selected from the group consisting of CH and N;
or a pharmaceutically acceptable salt thereof.

3. The compound of claim 1, wherein
R¹ is selected from the group consisting of R*-(3-hydroxy-phenyl), S*-(3-hydroxy-phenyl), 3-methoxyphenyl, R-(3-methoxyphenyl), S-(3-methoxyphenyl), R*-(3-methoxyphenyl), S*-(3-methoxyphenyl), R*-(3-methoxy-5-fluoro-phenyl) and S*-(3-methoxy-5-fluoro-phenyl);
Z is selected from the group consisting of CH and N;
R² and R³ are taken together with the atoms to which they are bound to form a fused ring structure selected from the group consisting of 1λ²-azepanyl, 1,4λ²-oxazonanyl, (Z)-2,3,6,9-tetrahydro-5H-1,4λ²-oxazoninyl, 1λ²,2λ²-diazepanyl and 4,7-dihydro-3H-1λ²,2λ²-diazepinyl;
Y is selected from the group consisting of CH and N;
or a pharmaceutically acceptable salt thereof.

4. The compound of claim 1, wherein
R¹ is selected from the group consisting R*-(3-hydroxy-phenyl), S*-(3-hydroxy-phenyl), R-(3-methoxy-phenyl), R*-(3-methoxy-phenyl), S*-(3-methoxy-phenyl), R*-(3-methoxy-5-fluoro-phenyl) and S*-(3-methoxy-5-fluoro-phenyl);
Z is CH;
R² and R³ are taken together with the atoms to which they are bound form 1λ²-azepanyl;
Y is selected from the group consisting of CH and N;
or a pharmaceutically acceptable salt thereof.

5. The compound of claim 1, wherein
R¹ is S-(3-methoxyphenyl),
Z is CH,
R² and R³ are taken together with the atoms to which they are bound to form 1,4λ²-oxazonanyl or (Z)-2,3,6,9-tetrahydro-5H-1,4λ²-oxazoninyl;
Y is selected from the group consisting of CH and N;
or a pharmaceutically acceptable salt thereof.

6. The compound of as in claim 1, wherein
R¹ is 3-methoxyphenyl;
Z is N;
R² and R³ are taken together with the atoms to which they are bound to form 1λ²,2λ²-diazepanyl or 4,7-dihydro-3H-1λ²,2λ²-diazepinyl;
Y is CH;
or a pharmaceutically acceptable salt thereof.

7. The compound of claim 1, selected from the group consisting of
(7R*)-7-(3-methoxyphenyl)-3-(1H-pyrazol-4-yl)-7,8,9,10,11,11a-hexahydro-5H-azepino[2,1-a]isoindol-5-one;
(Rac)-1-(3-methoxyphenyl)-8-(1H-pyrazol-4-yl)-1,2,3,4,5,5a-hexahydro-10H-[1,2]diazepino[7,1-a]isoindol-10-one;
(7S*)-7-(3-hydroxyphenyl)-3-(1H-pyrazol-4-yl)-7,8,9,10,11,11a-hexahydro-5H-azepino[2,1-a]isoindol-5-one;
and pharmaceutically acceptable salts thereof.

8. A pharmaceutical composition comprising a pharmaceutically acceptable carrier and a compound of claim 1.

9-10. (canceled)

11. A method of treating a disorder mediated by GRK2 activity, comprising administering to a subject in need thereof a therapeutically effective amount of the compound of claim 1.

12. The method of claim 11, wherein the disorder mediated by GRK2 activity is selected from the group consisting of obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD), n-stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m².

13. The method of claim 11, wherein the disorder mediated by GRK2 activity is selected from the group consisting of obesity, excess weight, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy, diabetic retinopathy, cardiac failure, cardiac hypertrophy, hypertension, angina, atherosclerosis, non-alcoholic steatohepatitis (NASH), non-alcoholic fatty liver disease (NAFLD), n-stage chronic kidney disease, chronic kidney disease, acute renal failure, and a measured GFR equal or greater than 125 mL/min/1.73 m²

14. The method of claim 11, wherein the disorder mediated by GRK2 activity is selected from the group consisting of obesity, excess weight, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy, diabetic retinopathy, non-alcoholic steatohepatitis (NASH), non-alcoholic fatty liver disease (NAFLD), n-stage chronic kidney disease, chronic kidney disease, acute renal failure, and a measured GFR equal or greater than 125 mL/min/1.73 m².

15-22. (canceled)
